SEQUENCE LISTING (44 sequences)

SEQ ID NO: 1 - protein, 11 aa, Artificial Sequence
  Albumin binding peptide (PEP)
DICLPRWGCLW

SEQ ID NO: 2 - protein, 5 aa, Artificial Sequence
  linker peptide
GGGGS

SEQ ID NO: 3 - protein, 6 aa, Artificial Sequence
  linker peptide
GGSGSS

SEQ ID NO: 4 - protein, 5 aa, Artificial Sequence
  linker peptide
EAAAK

SEQ ID NO: 5 - protein, 46 aa, Artificial Sequence
  linker peptide
AEAAAKEAAAKEAAAKEAAAKALEAEAAAKEAAAKEAAAKEAAAKA

SEQ ID NO: 6 - protein, 18 aa, Artificial Sequence
  linker peptide
KESGSVSSEQLAQFRSLD

SEQ ID NO: 7 - protein, 14 aa, Artificial Sequence
  linker peptide
EGKSSGSGSESKST

SEQ ID NO: 8 - protein, 12 aa, Artificial Sequence
  linker peptide
GSAGSAAGSGEF

SEQ ID NO: 9 - protein, 20 aa, Artificial Sequence
  E2A sequence
QCTNYALLKLAGDVESNPGP

SEQ ID NO: 10 - protein, 22 aa, Artificial Sequence
  F2A sequence
VKQTLNFDLLKLAGDVESNPGP

SEQ ID NO: 11 - protein, 18 aa, Artificial Sequence
  T2A sequence
EGRGSLLTCGDVEENPGP

SEQ ID NO: 12 - protein, 19 aa, Artificial Sequence
  P2A sequence
ATNFSLLKQAGDVEENPGP

SEQ ID NO: 13 - protein, 4 aa, Artificial Sequence
  caspase cleavage site
DEVD

SEQ ID NO: 14 - protein, 4 aa, Artificial Sequence
  caspase cleavage site
DLDV

SEQ ID NO: 15 - protein, 4 aa, Artificial Sequence
  caspase cleavage site
DEID

SEQ ID NO: 16 - protein, 4 aa, Artificial Sequence
  caspase cleavage site
DEHD

SEQ ID NO: 17 - protein, 4 aa, Artificial Sequence
  caspase cleavage site
DKAD

SEQ ID NO: 18 - protein, 4 aa, Artificial Sequence
  caspase cleavage site
DSFD

SEQ ID NO: 19 - protein, 4 aa, Artificial Sequence
  caspase cleavage site
DSSD

SEQ ID NO: 20 - protein, 4 aa, Artificial Sequence
  caspase cleavage site
DGKD

SEQ ID NO: 21 - protein, 4 aa, Artificial Sequence
  caspase cleavage site
DYND

SEQ ID NO: 22 - protein, 4 aa, Artificial Sequence
  caspase cleavage site
DRPD

SEQ ID NO: 23 - protein, 4 aa, Artificial Sequence
  caspase cleavage site
DNVD

SEQ ID NO: 24 - protein, 4 aa, Artificial Sequence
  caspase cleavage site
VQVD

SEQ ID NO: 25 - protein, 4 aa, Artificial Sequence
  caspase cleavage site
LETD

SEQ ID NO: 26 - protein, 4 aa, Artificial Sequence
  caspase cleavage site
LEHD

SEQ ID NO: 27 - protein, 4 aa, Artificial Sequence
  caspase cleavage site
WEHD

SEQ ID NO: 28 - protein, 6 aa, Artificial Sequence
  caspase cleavage site
ELQTDG

SEQ ID NO: 29 - protein, 6 aa, Artificial Sequence
  caspase cleavage site
RIEADS

SEQ ID NO: 30 - protein, 5 aa, Artificial Sequence
  caspase cleavage site
VDVAD

SEQ ID NO: 31 - protein, 4 aa, Artificial Sequence
  caspase cleavage site
DFRD

SEQ ID NO: 32 - protein, 6 aa, Artificial Sequence
  caspase cleavage site
KGDEVD

SEQ ID NO: 33 - protein, 6 aa, Artificial Sequence
  caspase cleavage site
RGDEVD

SEQ ID NO: 34 - protein, 11 aa, Artificial Sequence
  caspase cleavage site
CRGDCGGDEVD

SEQ ID NO: 35 - protein, 5 aa, Artificial Sequence
  caspase cleavage site
DEVDR

SEQ ID NO: 36 - protein, 10 aa, Artificial Sequence
  caspase cleavage site
CQRPPRDEVD

SEQ ID NO: 37 - protein, 4 aa, Artificial Sequence
  cathepsin cleavage site
GRRG

SEQ ID NO: 38 - protein, 4 aa, Artificial Sequence
  cathepsin cleavage site
FRRG

SEQ ID NO: 39 - protein, 4 aa, Artificial Sequence
  cathepsin cleavage site
ARRG

SEQ ID NO: 40 - protein, 5 aa, Artificial Sequence
  cathepsin cleavage site
KGRRG

SEQ ID NO: 41 - protein, 6 aa, Artificial Sequence
  cathepsin cleavage site
RGDRRG

SEQ ID NO: 42 - protein, 4 aa, Artificial Sequence
  caspase cleavage site
  VARIANT (2)..(3): Xaa may be any L- or D-type amino acid.
DXXD

SEQ ID NO: 43 - protein, 4 aa, Artificial Sequence
  caspase cleavage site
  VARIANT (2)..(3): Xaa may be any L- or D-type amino acid.
LXXD

SEQ ID NO: 44 - protein, 4 aa, Artificial Sequence
  caspase cleavage site
  VARIANT (2)..(3): Xaa may be any L- or D-type amino acid.
VXXD

(19) United States
(12) Patent Application Publication
(10) Pub. No.: US 2025/0302981 A1
(43) Pub. Date: Oct. 2, 2025

(54) NOVEL USE OF ANTICANCER AGENT PRODRUG CONJUGATE

(71) Applicants: UNIVERSITY OF ULSAN FOUNDATION FOR INDUSTRY COOPERATION, Ulsan (KR); PHAROSGEN, Seoul (KR); THE ASAN FOUNDATION, Seoul (KR); SEOUL NATIONAL UNIVERSITY R&DB FOUNDATION, Seoul (KR)

(72) Inventors: Sanghwan KIM, Gyeonggi-do (KR); Sang Yoon KIM, Seoul (KR); Youngro BYUN, Seoul (KR); Hanhee CHO, Seoul (KR)

(73) Assignees: UNIVERSITY OF ULSAN FOUNDATION FOR INDUSTRY COOPERATION, Ulsan (KR); PHAROSGEN, Seoul (KR); THE ASAN FOUNDATION, Seoul (KR); SEOUL NATIONAL UNIVERSITY R&DB FOUNDATION, Seoul (KR)

(21) Appl. No.: 18/858,714

(22) PCT Filed: Feb. 15, 2021
(86) PCT No.: PCT/KR2021/001925

§ 371 Date: Jun. 2, 2025

(30) Foreign Application Priority Data
Feb. 17, 2020 (KR) 10-2020-0019242

(51) Int. Cl. A61K 47/68; A61K 45/06; A61K 47/54; A61K 47/62; G01N 33/68

(52) U.S. Cl. CPC A61K 47/6835; A61K 45/06; A61K 47/545; A61K 47/62; G01N 33/6893; G01N 2333/4704; G01N 2800/52; G01N 2800/7028

(57) ABSTRACT

The present invention provides a pharmaceutical composition for the treatment of cancer having KRAS variant genotype or PTEN protein loss genotype, comprising an anti-cancer chemotherapeutic prodrug conjugate consisting of an albumin binding moiety, a linker, and an anti-cancer chemotherapeutic agent, for the effective treatment of cancers having KRAS variant genotype or PTEN protein loss genotype, which are known to be refractory cancers.

### CROSS-REFERENCE TO RELATED APPLICATIONS

The present application is the U.S. National Stage of International Patent Application No. PCT/KR2021/001925, filed Feb. 15, 2021, and claims priority to South Korean Patent Application No. 10-2020-0019242, filed Feb. 17, 2020.

### SEQUENCE LISTING

The present application contains a Sequence Listing which has been submitted electronically in ASCII format and is hereby incorporated by reference in its entirety. Said ASCII file, created May 28, 2025, is named “122257-0123_SL.txt” and is 9,259 bytes in size.

### TECHNICAL FIELD

The present invention relates to novel uses of anti-cancer prodrugs, and more specifically to uses of anti-cancer prodrugs that are specific for cancers with KRAS variants or PTEN protein loss genotypes.

### BACKGROUND

Anticancer chemotherapeutic agents (or anticancer drugs) are the mainstay of cancer treatment due to their potent anti-cancer effects, but they also present challenges such as severe side effects and dosing restrictions due to toxicity. Therefore, it is necessary to ensure that these therapies do not act on normal tissues and selectively act on cancerous tissues. To this end, drugs that selectively deliver drugs to cancerous tissues by using antibodies or peptides that recognize and bind to biomarkers that are not expressed or rarely expressed in normal cells but are specifically expressed in tumor cells have been commonly applied. However, these drugs have the limitation that they can only be used for patients who have developed cancer with the biomarker, as the genotype of the biomarker varies from patient to patient even within the same cancer type.

In addition, recent studies have revealed intratumor heterogeneity, referring to the presence of diverse genotypes within a single cancer tissue. Consequently, it has been recognized that a biopsy, which is typically used to characterize the cancer, may not accurately represent the tumor's overall genotype. This implies that even if a specific biomarker is detected, the effectiveness of drugs targeting that biomarker remains uncertain.

Moreover, even if the tumor cells expressing the biomarker are in the majority in the cancer tissue, the drug may not affect the remaining tumor cells, resulting in recurrence of cancer tissue growth due to the residual tumor cells after treatment. For these reasons, conventional drugs have fundamental limitations in selectively delivering anticancer drugs to all tumor cells, and are not ideal delivery systems for anticancer chemotherapeutics. In addition, if most cancer tissues express the corresponding biomarker, they may affect other cells in close proximity to or adjacent to the cancer tissue, therefore causing side effects or toxicity.

To address these issues, prodrugs have been developed to enable specific activation within tumor cells and cancer tissues. With respect to these technologies, U.S. Pat. No. 7,445,764 discloses a conjugate of cleavable matrixmetalloprotease (MMP), and a conjugate of cleavable peptide and doxorubicin anticancer drug. Also, U.S. Patent Publication No. 2010/0111866 discloses a prodrug conjugate in the form of maleimide-hydrazone-doxorubicin, and U.S. Patent Publication No. 2013/0338422 discloses a prodrug conjugate in the form of peptide sequence-DEVD-doxorubicin. However, these conjugates still face challenges such as low potency, low selectivity, and frequent dosing.

Therefore, there is a need to develop chemotherapeutic agents such as prodrugs that selectively act on tumor cells and do not affect normal tissues, thereby minimizing side effects and providing amplified efficacy.

### DETAILED DESCRIPTION OF THE INVENTION

### Technical Problem

The present invention can solve a variety of problems including those mentioned above, and it is an object of the present invention to provide an anticancer chemotherapeutic prodrug conjugate that selectively acts on tumor cells with KRAS variants or PTEN protein loss genotypes but has no effect on normal tissue, thereby enabling amplified efficacy and minimal side effects. It is also an object of the present invention to provide uses for the anticancer chemotherapeutic prodrug conjugate in the treatment of various cancers. However, these problems are exemplary and are not intended to limit the scope of the present invention.

### Technical Solution

In one aspect of the present invention, there is provided a pharmaceutical composition for the treatment of cancer having a KRAS variant or PTEN protein loss genotype comprising a chemotherapeutic prodrug conjugate comprising albumin binding moiety, linker, and chemotherapeutic agent.

In another aspect of the present invention, there is provided method of providing the best prescription for a cancer patient, comprising:
isolating DNA or protein during biopsy from a cancer tissue obtained from the cancer patient;
identifying the genotype of the gene encoding KRAS and PTEN protein or investigating the loss of expression of the PTEN protein using the isolated DNA or protein; and,
determining the cancer patient as a subject of administration of an anticancer prodrug conjugate comprising albumin binding moiety, linker, and anticancer compound if it is found that the patient's gene encoding KRAS has been mutated, or the gene encoding the PTEN protein has been mutated such that it causes a loss of the PTEN protein, or that the loss of the PTEN protein is identified.

In another aspect of the present invention, there is provided a method of treating cancer in a cancer patient with KRAS variant or the loss of PTEN protein, comprising:
isolating DNA or protein from a cancer tissue biopsy obtained from the cancer patient; identifying the genotype of the gene encoding KRAS and PTEN protein or investigating the loss of expression of the PTEN protein using the isolated DNA or protein; and,
administering therapeutically effective amount of an anticancer prodrug conjugate comprising albumin binding moiety, linker, and anticancer compound to the cancer patient if it is found that the patient's gene encoding the KRAS has been mutated, or the gene encoding the PTEN protein has been mutated such that it causes a loss of the PTEN protein, or that the loss of the PTEN protein is identified.

In another aspect of the present invention, there is provided a use of a chemotherapeutic prodrug conjugate comprising albumin binding moiety, linker, and chemotherapeutic agent for the preparation of a targeted therapeutic agent for a cancer having KRAS variant or PTEN protein loss genotype.

### Effect of the Invention

According to one embodiment of the present invention made as described above, it is possible to effectively treat chemotherapy-resistant cancers with genotypes of KRAS variants or loss of PTEN protein that have been difficult to treat. However, this effect is not intended to limit the scope of the present invention.

### BRIEF DESCRIPTION OF THE DRAWINGS

FIG. 1 shows the result of HPLC analysis after reaction of maleimide-KGDEVD-PABC-doxorubicin according to one embodiment of the present invention with commercially available human serum albumin (HSA).

FIG. 2 shows the results of HPLC analysis after reaction of maleimide-KGDEVD-PABC-doxorubicin according to one embodiment of the present invention with human plasma.

FIG. 3 shows the result of HPLC analysis related to the cleavage reaction of human serum albumin-conjugated maleimide-KGDEVD-PABC-doxorubicin (HSA-KGDEVD-PABC-doxorubicin) by recombinant human caspase-3 (KGDEVD corresponds to SEQ ID NO: 32).

FIG. 4 shows the result of HPLC analysis related to the cleavage reaction of human serum albumin-conjugated maleimide-KGDVED-PABC-doxorubicin (HSA-KGDVED-PABC-doxorubicin) by recombinant human caspase-3 (KGDEVD corresponds to SEQ ID NO: 32).

FIG. 5 is a graph showing the in vitro concentration dependence of human serum albumin-conjugated maleimide-KGDEVD-PABC-doxorubicin (HSA-KGDEVD-PABC-doxorubicin) in the presence and absence of the recombinant human caspase-3 (KGDEVD corresponds to SEQ ID NO: 32).

FIG. 6 is a graph showing the change in plasma concentration of doxorubicin over time after intravenous administration of maleimide-KGDEVD-PABC-doxorubicin or AcKGDEVD-PABC-doxorubicin according to one embodiment of the present invention to Sprague-Dawley rats (KGDEVD corresponds to SEQ ID NO: 32).

FIG. 7 is a graph showing the change in plasma concentration of doxorubicin after intravenous administration of maleimide-KGDEVD-PABC-doxorubicin or AcKGDEVD-PABC-doxorubicin according to one embodiment of the present invention to cynomolgus monkeys (KGDEVD corresponds to SEQ ID NO: 32).

FIG. 8 shows the result of western blot to identify the group of cancer cells with PTEN protein loss genotype.

FIG. 9 is a series of fluorescence micrographs showing the results of administering fluorescently labeled human serum albumin to tumor cells with wild-type KRAS genotype.

FIG. 10 is a series of fluorescence micrographs showing the results of administering fluorescently labeled human serum albumin to tumor cells with KRAS variant genotype.

FIG. 11 is a series of fluorescence micrographs showing the results of administration of human serum albumin-conjugated maleimide-KGDEVD-PABC-doxorubicin prodrug conjugate according to one embodiment of the present invention to wild-type KRAS tumor cells and tumor cells with KRAS variant genotype, respectively (KGDEVD corresponds to SEQ ID NO: 32).

FIG. 12 is a series of fluorescence micrographs showing the results of administration of fluorescently labeled human serum albumin to tumor cells of the wild-type PTEN protein genotype.

FIG. 13 is a series of fluorescence micrographs showing the results of administration of fluorescently labeled human serum albumin to tumor cells of the PTEN protein loss genotype.

FIG. 14 is a series of graphs showing the results of in vitro experiments where the tumor cells of wild-type KRAS or KRAS variant genotypes were treated with maleimide-KGDEVD-PABC-doxorubicin according to one embodiment of the present invention to determine concentration-dependent cytotoxicity (KGDEVD corresponds to SEQ ID NO: 32).

FIG. 15 is a series of graphs (top) showing the results of in vitro experiments where the tumor cells of wild-type KRAS or KRAS variant genotypes were treated with maleimide-KGDEVD-PABC-doxorubicin according to one embodiment of the present invention to determine concentration-dependent cytotoxicity, and a chart (bottom) showing the type of cells used in the experiment, genotypes (KRAS and PTEN), and measured IC₅₀ level of doxorubicin (KGDEVD corresponds to SEQ ID NO: 32).

FIG. 16 is a series of graphs showing the results of investigating tumor sizes depending on the concentration of maleimide-KGDEVD-PABC-doxorubicin according to one embodiment of the present invention administrated to mice that were inoculated with tumor cells of wild-type KRAS or KRAS variant genotypes (KGDEVD corresponds to SEQ ID NO: 32).

FIG. 17 is a series of graphs showing the results of investigating weights of mice depending on the concentration of maleimide-KGDEVD-PABC-doxorubicin according to one embodiment of the present invention administrated to the mice, which were inoculated with tumor cells of wild-type KRAS or KRAS variant genotypes (KGDEVD corresponds to SEQ ID NO: 32).

FIG. 18 is a graph showing the results of investigating tumor sizes (left) and weights of mice (right) depending on the concentration of maleimide-KGDEVD-PABC-doxorubicin according to one embodiment of the present invention administrated to the mice, which were inoculated with tumor cells of the PTEN loss genotype (H1299) (KGDEVD corresponds to SEQ ID NO: 32).

FIG. 19 is a graph showing the results of investigating tumor sizes (left) and weights of mice (right) depending on the concentration of maleimide-KGDEVD-PABC-doxorubicin according to one embodiment of the present invention administrated to the mice inoculated with tumor cells of the KRAS variant genotype, to compare the maleimide-KGDEVD-PABC-doxorubicin (MDP1) with commercial Aldoxorubicin (Aldox) (KGDEVD corresponds to SEQ ID NO: 32).

FIG. 20 is a series of photographs of the tumor tissues obtained from mice inoculated with tumor cells of the KRAS variant genotype (left) and the graph showing the weights of tumor tissues (right) to compare the maleimide-KGDEVD-PABC-doxorubicin (MDP1) according to one embodiment of the present invention with commercial Aldoxorubicin (Aldox) (KGDEVD corresponds to SEQ ID NO: 32).

### BEST MODE FOR THE INVENTION

### Definitions of Terms

As used herein, the term “albumin binding moiety” refers to a functional group that can bind, either covalently or non-covalently, to plasma albumin in vivo. An example of such an albumin binding moiety is maleimide, which let the compound including it form a stable albumin-compound conjugate in plasma by covalent bonding between a free thiol group of cysteine, the 34^{th} amino acid of plasma albumin, and the maleimide group. In addition, 4-(p-iodophenyl)butyric acid is also known to selectively bind to albumin by a non-covalent bond. Besides, oleate, folate, and palmitic acid (PA) are known to bind non-covalently to various sites on albumin, and albumin-binding peptide (PEP, DICLPRWGCLW, SEQ ID NO: 1) is also known to selectively bind to specific sites on albumin by non-covalent bonding. Furthermore, various compounds are known to bind specifically or non-specifically to albumin (Zorzi et al., Med. Chem. Commun. 2019, 10(7): 1068-1081)

As used herein, the term “linker” refers to a structure that connects two compounds, and there are two main types of linkers: peptide linkers and non-peptide linkers. Peptide linkers are divided into in vivo cleavable peptide linkers and in vivo non-cleavable linkers, and representative of the in vivo non-cleavable peptide linkers are (G4S)ₙ(repeat unit: SEQ ID NO: 2), (GGSGSS)ₙ (repeat unit: SEQ ID NO: 3), (EAAAK)ₙ(repeat unit: SEQ ID NO: 4), A(EAAAK)ₙA, A(EAAAK)₄ALEA(EAAAK)₄A (SEQ ID NO: 5), KESGSVSSEQLAQFRSLD (SEQ ID NO: 6), EGKSSGSGSESKST (SEQ ID NO: 7), GSAGSAAGSGEF (SEQ ID NO: 8), (Ala-Pro)ₙ and the like, and in vivo cleavable peptide linkers include cyclopeptide linkers and protease-sensitive linkers. The protease-sensitive linker is a peptide having a cleavage site that is cleaved by a protease present in vivo, such as a caspase, cathepsin, purine, or matrix metalloprotease (MMP). These protease-sensitive linkers may be E2A (SEQ ID NO: 9), F2A (SEQ ID NO: 10), T2A (SEQ ID NO: 11), or P2A (SEQ ID NO: 12) peptides; or, caspase- or cathepsin-dependent cleavage sites such as DEVD (SEQ ID NO: 13), DLDV (SEQ ID NO: 14), DEID (SEQ ID NO: 15), DEHD (SEQ ID NO: 16), DKAD (SEQ ID NO: 17), DSFD (SEQ ID NO: 18), DSSD (SEQ ID NO: 19), DGKD (SEQ ID NO: 20), DYND (SEQ ID NO: 21), DRPD (SEQ ID NO: 22), DNVD (SEQ ID NO: 23), VQVD (SEQ ID NO: 24), LETD (SEQ ID NO: 25), LEHD (SEQ ID NO: 26), WEHD (SEQ ID NO: 27), ELQTDG (SEQ ID NO: 28), RIEADS (SEQ ID NO: 29), VDVAD (SEQ ID NO: 30), DFRD (SEQ ID NO: 31), KGDEVD (SEQ ID NO: 32), RGDEVD (SEQ ID NO: 33), CRGDCGGDEVD (SEQ ID NO: 34), DEVDR (SEQ ID NO: 35), CQRPPRDEVD (SEQ ID NO: 36), GRRG (SEQ ID NO: 37), FRRG (SEQ ID NO: 38), ARRG (SEQ ID NO: 39), KGRRG (SEQ ID NO: 40), RGDRRG (SEQ ID NO: 41), DXXD (SEQ ID NO: 42), LXXD (SEQ ID NO: 43), or VXXD (SEQ ID NO: 44). Non-peptide linkers include alkylene linkers, polyalkylene oxide linkers, NHS ester linkers, arylene linkers, p-aminocarbamate (PABC) linkers, Merrifield linkers, Wang linkers, Sasrin linkers, Tritiyl linkers, RINK-amide linkers, and Kenner linkers, Silyl linkers, Triazene linkers, photocleavable linkers, maleimide alkane linkers, hydrazone linkers, disulfide linkers, glucuronide-MABC linkers, azobenzene linkers, dialkoxydiphenylsilane linkers, Val-Cit-PABC linkers, etc.

As used herein, the term “KRAS variant” refers to an abnormal K-Ras protein produced by a mutation in the KRAS (Kirsten rat sarcoma viral oncogene homolog) gene, one of the oncogenes. These KRAS variants contain a substitution of glycine (G), the 12th amino acid of wild-type KRAS, with aspartic acid (D), cysteine (C), serine (S), or valine (V). These KRAS variants are always active and stimulate cell proliferation, even in the absence of signaling from EGFR or other tyrosine kinase proteins. In these cancers, anticancer drugs that target EGFR, such as Herceptin, become ineffective. KRAS mutations account for approximately 15 to 20% of human cancers, particularly in pancreatic, colorectal, lung cancers, and leukemia. Notably, it is known that about 30 to 40% of colorectal cancers and 15 to 30% of lung cancers carry KRAS variants.

As used herein, the term “PTEN protein” stands for phosphatase and tension homolog and is encoded by the PTEN gene, whose mutations have been shown to be an important step in the development of many cancers. PTEN is known as a tumor suppressor gene by acting on phosphatases involved in the regulation of the cell cycle. It can act as a tumor suppressor gene by negatively regulating intracellular levels of phosphatidylinositol-3,4,5-triphosphate and Akt/PKB signaling pathway. During tumor development, the deletion or mutation of PTEN leads to increased cell proliferation and decreased apoptosis. It has been reported that one copy of the PTEN gene is deleted in 70% of prostate cancer patients.

As used herein, the term “anticancer prodrug conjugate” refers to a conjugate in which an anticancer compound is linked by a linker to another compound, protein, or peptide. This conjugate either lacks pharmacological activity in its initial state or travels through the bloodstream with an increased half-life to release the active form of the anticancer compound upon cleavage of the linker near the lesion site.

As used herein, the term “PABC” refers to p-aminocarbamate. An antibody-drug conjugate linked via PABC is stable in the bloodstream but is selectively cleaved by an intracellular protease within the lysosome after entering a cell.

### Detailed Description of the Invention

In one aspect of the present invention, there is provided a pharmaceutical composition for treating a cancer having a KRAS variant or PTEN protein loss genotype, comprising an anti-cancer chemotherapeutic prodrug conjugate consisting of albumin binding moiety, linker, and anti-cancer chemotherapeutic agent.

In the pharmaceutical composition, the albumin binding moiety may be selected from a maleimide group, a pyridyldithiol group, an oleate group, a folate group, an albumin binding peptide (PEP, SEQ ID NO: 1), a palmitate group, 4-(p-iodophenyl)butyric acid, or a single chain-based antibody analog that specifically binds to albumin, such as V_{H}H, scFv, V_{NAR}, DARPin, nanobody, monobody, or VLR.

In the pharmaceutical composition, the linker may be a peptide linker, non-peptide linker, or a mixture of the peptide linker and non-peptide linker. Here, the peptide linker may be an in vivo cleavable peptide linker or an in vivo non-cleavable linker, wherein the in vivo cleavable peptide may be a cyclopeptide peptide linker or a protease-sensitive peptide linker. The protease-sensitive peptide linker may be a peptide that can be cleaved by a caspase, cathepsin, purine, or matrix metalloprotease, and more specifically, it may be DEVD (SEQ ID NO: 13), DLDV (SEQ ID NO: 14), DEID (SEQ ID NO: 15), DEHD (SEQ ID NO: 16), DKAD (SEQ ID NO: 17), DSFD (SEQ ID NO: 18), DSSD (SEQ ID NO: 19), DGKD (SEQ ID NO: 20), DYND (SEQ ID NO: 21), DRPD (SEQ ID NO: 22), DNVD (SEQ ID NO: 23), VQVD (SEQ ID NO: 24), LETD (SEQ ID NO: 25), LEHD (SEQ ID NO: 26), WEHD (SEQ ID NO: 27), ELQTDG (SEQ ID NO: 28), RIEADS (SEQ ID NO: 29), VDVAD (SEQ ID NO: 30), DFRD (SEQ ID NO: 31), KGDEVD (SEQ ID NO: 32), RGDEVD (SEQ ID NO: 33), CRGDCGGDEVD (SEQ ID NO: 34), DEVDR (SEQ ID NO: 35), CQRPPRDEVD (SEQ ID NO: 36), GRRG (SEQ ID NO: 37), FRRG (SEQ ID NO: 38), ARRG (SEQ ID NO: 39), KGRRG (SEQ ID NO: 40), RGDRRG (SEQ ID NO: 41), DXXD (SEQ ID NO: 42), LXXD (SEQ ID NO: 43), or VXXD (SEQ ID NO: 44). The linker in the form of a mixture of the peptide linker and non-peptide linker as mentioned above may be KGDEVD (SEQ ID NO: 32)-PABC, DEVD (SEQ ID NO: 13)-PABC, RGDEVD (SEQ ID NO: 33)-PABC, RGDEVD (SEQ ID NO: 33)-MBA, CQRPPRDEVD (SEQ ID NO: 36)-PABC, DEID (SEQ ID NO: 15)-PABC, DLVD (SEQ ID NO: 14)-PABC, RGDEVD (SEQ ID NO: 33)-MBA, or KGDEVD (SEQ ID NO: 32)-PABC.

In the pharmaceutical composition, the anticancer chemotherapeutic agent may be selected from the group consisting of cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, and derivatives thereof.

In a more specific embodiment, the conjugate may be selected from the group consisting of maleimide-KGDEVD-PABC-doxorubicin, maleimide-KGDEVD-PABC-daunorubicin, maleimide-KGDEVD-PABC-paclitaxel, maleimide-KGDEVD-PABC-MMAE, maleimide-DEVD-PABC-doxorubicin, maleimide-DEID-PABC-doxorubicin, maleimide-DLVD-PABC-doxorubicin, maleimide-DEVD-doxorubicin, pyridyldithiol-KGDEVD-PABC-doxorubicin, oleate-KGDEVD-PABC-doxorubicin, folate-KGDEVD-PABC-doxorubicin, and HSA-maleimide-KGDEVD-PABC-doxorubicin, wherein KGDEVD corresponds to SEQ ID NO:32, DEVD corresponds to SEQ ID NO: 13, DEID corresponds to SEQ ID NO: 15, and DLVD corresponds to SEQ ID NO:14.

In another aspect of the present invention, there is provided a method of providing information for the prescription for a cancer patient, comprising
isolating DNA or protein during biopsy from a cancer tissue obtained from the cancer patient;
identifying the genotype of the gene encoding KRAS and PTEN protein or investigating the loss of expression of the PTEN protein using the isolated DNA or protein; and,
determining the cancer patient as a subject of administration of an anticancer prodrug conjugate comprising albumin binding moiety, linker, and anticancer compound if it is found that the patient's gene encoding KRAS has been mutated, or the gene encoding the PTEN protein has been mutated such that it causes a loss of the PTEN protein, or that the loss of the PTEN protein is identified.

In another aspect of the present invention, there is provided method of providing the best prescription for a cancer patient, comprising
isolating DNA or protein during biopsy from a cancer tissue obtained from the cancer patient;
identifying the genotype of the gene encoding KRAS and PTEN protein or investigating the loss of expression of the PTEN protein using the isolated DNA or protein; and
determining the cancer patient as a subject of administration of an anticancer prodrug conjugate comprising albumin binding moiety, linker, and anticancer compound if it is found that the patient's gene encoding KRAS has been mutated, or the gene encoding the PTEN protein has been mutated such that it causes a loss of the PTEN protein, or that the loss of the PTEN protein is identified.

In another aspect of the present invention, there is provided a method of providing a cancer patient with the best prescription, comprising isolating DNA or protein during biopsy from a cancer tissue obtained from the cancer patient; identifying the genotype of the gene encoding KRAS and PTEN protein or investigating the loss of expression of the PTEN protein; and determining the cancer patient as a subject of administration of an anticancer prodrug conjugate comprising albumin binding moiety, linker, and anticancer compound if it is found that the patient's gene encoding KRAS has been mutated, or the gene encoding the PTEN protein has been mutated such that it causes a loss of the PTEN protein, or that the loss of the PTEN protein is identified.

In the methods, the presence or absence of variation in the gene encoding KRAS may be analyzed by sequencing, DNA microarray, or allele-specific PCR reaction.

In the methods, the determination of the loss of PTEN protein may be performed by genotyping the gene encoding the PTEN protein or by protein quantification of the expression of the PTEN protein. The genotyping can be performed by any method known in the art, such as sequencing, DNA microarray, allele-specific PCR, as described above, and the protein quantification can be performed by any method known in the art, such as mass spectrometry, western blot analysis, protein microarray analysis, ELISA, RIA, immunoprecipitation, etc. More preferably, when used in clinical practice, it can be performed using mass spectrometry.

In another aspect of the present invention, there is provided a method of treating cancer in a cancer patient with KRAS variant or the loss of PTEN protein, comprising
isolating DNA or protein from a cancer tissue biopsy obtained from the cancer patient;
identifying the genotype of the gene encoding KRAS and PTEN protein or investigating the loss of expression of the PTEN protein using the isolated DNA or protein; and,
administering therapeutically effective amount of an anticancer prodrug conjugate comprising albumin binding moiety, linker, and anticancer compound to the cancer patient if it is found that the patient's gene encoding the KRAS has been mutated, or the gene encoding the PTEN protein has been mutated such that it causes a loss of the PTEN protein, or that the loss of the PTEN protein is identified

In the above therapeutic method, the presence of a mutation in the gene encoding KRAS may be analyzed by sequencing, DNA microarray, or allele-specific PCR reaction.

In the above therapeutic method, the determination of loss of PTEN protein may be performed by genotyping of the gene encoding the PTEN protein or by protein quantification of the expression of the PTEN protein. Furthermore, the genotyping may be performed by any method known in the art, such as sequencing, DNA microarray analysis, allele-specific PCR, as described above, and the protein quantification may be performed by any method known in the art, such as mass spectrometry, western blot analysis, protein microarray analysis, ELISA, RIA, immunoprecipitation, etc. More preferably, when used in clinical practice, it can be performed using mass spectrometry.

In another aspect of the present invention, there is provided a chemotherapeutic prodrug conjugate comprising albumin binding moiety, linker, and chemotherapeutic agent for preparation of a targeted therapeutic agent for a cancer having KRAS variant or PTEN protein loss genotype.

The present invention will be described in more detail below.

### Chemotherapeutic Prodrug Conjugate (Chemotherapeutic Prodrug Conjugate)

The anticancer chemotherapeutic prodrug conjugate presented herein comprise (i) an albumin-binding moiety, (ii) a peptide linker that is linked to the albumin binding moiety directly or by another linker and can be cleaved by caspase or cathepsin, and (iii) an anticancer chemotherapeutic agent linked to the peptide linker directly or by another linker. As described hereinafter, the conjugate of the present invention is useful in methods of inducing cell death, amplifying cell death, and treating cancer.

Detailed preferred embodiments of the anticancer chemotherapeutic prodrug conjugate of the present invention are as follows:

Maleimide-KGDEVD-PABC-Doxorubicin, Maleimide-KGDEVD-PABC-Daunorubicin, Maleimide-KGDEVD-PABC-Paclitaxel, Maleimide-KGDEVD-PABC-MMAE, Maleimide-DEVD-PABC-Doxorubicin, Maleimide-DEID-PABC-Doxorubicin, Maleimide-DLVD-PABC-Doxorubicin, Maleimide-DEVD-doxorubicin, Maleimide-DEVD-MMAE, Pyridyldithiol-KGDEVD-PABC-doxorubicin, Oleate-KGDEVD-PABC-doxorubicin, Folate-KGDEVD-PABC-doxorubicin, Maleimide-KGRRG-PABC-doxorubicin, Maleimide-KGRRG-PABC-daunorubicin, Maleimide-KGRRG-PABC-paclitaxel, Maleimide-KGRRG-PABC-MMAE, Maleimide-GRRG-PABC-doxorubicin, Maleimide-FRRG-PABC-doxorubicin, Maleimide-ARRG-PABC-doxorubicin, Maleimide-GRRG-doxorubicin, Maleimide-GRRG-MMAE, Pyridyldithiol-KGRRG-PABC-doxorubicin, Oleate-KGRRG-PABC-doxorubicin, and Folate-KGRRG-PABC-doxorubicin, wherein KGDEVD corresponds to SEQ ID NO:32, DEVD corresponds to SEQ ID NO: 13, DEID corresponds to SEQ ID NO: 15, and DLVD corresponds to SEQ ID NO:14, KGRRG corresponds to SEQ ID NO:40, GRRG corresponds to SEQ ID NO:37, FRRG corresponds to SEQ ID NO:38, and ARRG corresponds to SEQ ID NO:39.

The individual anticancer chemotherapeutic prodrug conjugates are described in detail in the embodiments provided below.

### Albumin Binding Moiety

As mentioned above, the anticancer chemotherapeutic prodrug conjugates presented herein comprise an albumin binding moiety. The albumin binding moiety binds to serum albumin. According to one embodiment, the albumin binding moiety comprises one or more of a functional chemosensory group, a peptide functional group, an antibody functional group (including antibody fragment and short-chain antibody functional group), an aptamer, an oligonucleotide, or a saccharide. Upon in vivo administration, the albumin binding moiety can bind to serum albumin and passively target tumor tissue by enhanced permeability and retention (EPR). Furthermore, the albumin binding moiety can selectively target tumor tissue having KRAS variant or PTEN protein loss genotype.

In a more preferred embodiment, the albumin binding moiety may be a maleimide group, a pyridyldithiol group, an oleate group, a polyethylene glycol (PEG) group, a folate group, a palmitate group, an albumin-binding peptide (PEP, SEQ ID NO: 1), or a single chain-based antibody fragment or its analogue that specifically binds to albumin, such as scFv, V_{H}H, nanobody, monobody, V_{NAR}, affibody, VLR, etc.

### Linker

As mentioned above, the prodrug conjugates presented herein comprise a linker connecting the chemotherapeutic agent to the albumin binding moiety. The linker may be a peptide linker or a non-peptide linker, optionally in a form comprising both the peptide linker and non-peptide linker. The peptide linker may be an in vivo cleavable peptide linker or an in vivo non-cleavable linker, wherein the in vivo cleavable peptide may be a cyclopeptide peptide linker or a protease-sensitive peptide linker. The protease-sensitive peptide linker may be a peptide that can be cleaved by caspase, cathepsin, purine, or metalloprotease, comprising a caspase-cleavable linker or a cathepsin-cleavable linker.

As used herein, the term “caspase” refers to a cysteine-aspartic protease, i.e., a cysteine-dependent aspartate-directed protease which is activated (e.g., expressed) by the progression of cell death. According to a preferred embodiment, the caspase may be caspase-3, caspase-7 or caspase-9.

As used herein, the term “cathepsin” refers to a protease that is overexpressed in a tumor cell and is activated in an acidic environment, such as a lysosome. According to a preferred embodiment, the cathepsin may be cathepsin-B or cathepsin-D.

As used herein, the term “amino acid” refers to an amino acid including a naturally occurring amino acid (natural amino acid) and a non-naturally occurring amino acid which is a chemically processed derivative of a naturally occurring amino acid (synthetic amino acid).

Natural amino acids, except for glycine, contain a chiral carbon atom. Furthermore, the amino acids can be in the form of L- or D-isomers. Preferably, the amino acids comprise β-alanine (BALA), γ-aminobutyric acid (GABA), 5-aminovaleric acid, glycine (Gly or G), phenylglycine, arginine (Arg or R), homoarginine (Har or hR), alanine (Ala or A), valine (Val or V), norvaline, leucine (Leu or L), norleucine (Nle), and isoleucine (Ile or I), serine (Ser or S), isoserine, homoserine (Hse), threonine (Thr or T), allothreonine, methionine (Met or M), ethionine, glutamic acid (Glu or E), aspartic acid (Asp or D), asparagine (Asn or N), cysteine (Cys or C), cystine, phenylalanine (Phe or F), tyrosine (Tyr or Y), tryptophan (Trp or W), lysine (Lys or K), hydroxylysine (Hyl), histidine (His or H), ornithine (Orn), glutamine (Gln or Q), citrulline, proline (Pro or P), and 4-hydroxyproline (Hyp or O).

As used herein, the term “peptide” refers to a peptide or its analogues (peptide analogues), wherein the peptide analogs include naturally occurring amino acids and modified non-naturally occurring amino acids with glycosylation, modified R functional groups, and/or modified peptide main chains. According to a preferred embodiment, the peptide comprises only the L-isomers of a chiral amino acid. According to another embodiment, the peptide comprises only the D-isomers of a chiral amino acid. According to another embodiment, the peptide comprises one or more of both the L-isomers and D-isomers of a chiral amino acid.

The peptide analogue may include, within the amino acid sequence of the peptide, an amide bond such as a urethane, urea, ester, or thioester bond, as well as at least one other bond. The peptide or peptide analogue referred to herein may be linear, cyclic or branched, but preferably linear.

As used herein, the term “caspase-cleavable peptide linker” refers to a peptide sequence having two or more amino acid residues that are cleavable by a caspase. In some embodiments, the caspase-cleavable peptide linker can be cleaved by a peptide comprising the amino acid sequence of Asp-Xaa-Xaa-Asp (SEQ ID NO: 42), such as caspase-3 or caspase-7, wherein the Xaa comprises any amino acid in the L- or D-isomers. In some other embodiments, the caspase-cleavable peptide linker can be cleaved by a peptide comprising the amino acid sequence Leu-Xaa-Xaa-Asp (SEQ ID NO: 43) or Val-Xaa-Xaa-Asp (SEQ ID NO: 44), such as caspase-9, wherein the Xaa comprises any amino acid in the L- or D-isomers.

As used herein, the term “cathepsin-cleavable peptide linker” refers to a peptide sequence having two or more amino acid residues that are cleavable by cathepsin. In some embodiments, the cathepsin-cleavable peptide linker can be cleaved by a peptide comprising the amino acid sequence Xaa-Arg-Arg-Xaa (SEQ ID NO: 49), such as cathepsin-B, wherein the Xaa comprises any L-amino acid.

According to a preferred embodiment, the caspase-cleavable peptide linker can be selected from the group consisting of:

(SEQ ID NO: 13)
Asp-Glu-Val-Asp,
(SEQ ID NO: 14)
Asp-Leu-Val-Asp,
(SEQ ID NO: 15)
Asp-Glu-Ile-Asp,
and
(SEQ ID NO: 16)
Leu-Glu-His-Asp.

According to a more preferred embodiment, the caspase-cleavable peptide linker comprises

(SEQ ID NO: 32)
Lys-Gly-Asp-Glu-Val-Asp.

According to a preferred embodiment, the cathepsin-cleavable peptide linker can be selected from the group consisting of:

(SEQ ID NO: 37)
Gly-Arg-Arg-Gly,
(SEQ ID NO: 38)
Phe-Arg-Arg-Gly,
and
(SEQ ID NO: 39)
Ala-Arg-Arg-Gly.

According to a more preferred embodiment, the cathepsin-cleavable peptide linker comprises Lys-Gly-Arg-Arg-Gly (SEQ ID NO: 40).

In some embodiments, the presence of the caspase- or cathepsin-cleavable peptide linker renders the prodrug conjugate inactive until the linker is cleaved. Consistent with these examples, the prodrug conjugate causes minimal harm to healthy cells. This is because the prodrug conjugate according to one embodiment of the present invention can be activated only in the presence of caspase or cathepsin. This is important because normal cells also undergo apoptosis just like cancer cells. Thus, in some embodiments, the chemotherapeutic prodrug conjugate according to one embodiment of the invention exhibit minimal side effects.

Optionally, the chemotherapeutic prodrug conjugate according to one embodiment of the present invention may comprise a non-cleavable peptide instead of the peptide linker cleavable by a protease. Chemotherapeutic prodrug conjugates comprising such non-cleavable peptides, although somewhat less active than those employing cleavable peptides, exhibited greater anticancer activity than chemotherapeutic agents alone in cancers with KRAS variant or PTEN protein loss genotype. This could be attributed to the fact that even though it is a non-cleavable peptide, when the chemotherapeutic prodrug conjugate according to one embodiment of the present invention is taken up into cancer cells by endocytosis and then degraded through the endosome→lysome pathway, the chemotherapeutic agent is released by non-specific protein cleavage and translocated into the nucleus to finally destroy the cancer cells.

Therefore, the present invention does not exclude the use of flexible linkers such as (GS₄)ₙ that are commonly used in the preparation of conventional fusion proteins, instead of the peptide linkers cleavable by proteases. Furthermore, it does not exclude the use of chemical linkers that connect the chemotherapeutic agent and maleimide group by covalent bonds other than peptide bonds, as long as they can be cleaved within a cell.

Such chemical linkers are not particularly limited by the present invention and may be any linker that is used for the purpose of binding in pharmaceutical compositions. p-aminocarbamate (PABC) linkers can be suggested as a preferred linker, but it is not limited to this example only but can include alkylene linkers, arylene linkers, polyalkylene oxide linkers (e.g., PEG), NHS ester linkers, Merrifield linkers, Wang linkers, Sasrin linkers, Tritiyl linkers, RINK-amide linkers, Kenner linkers, Silyl linkers, Triazene linkers, photocleavable linkers, maleimide alkane linkers, hydrazone linkers, disulfide linkers, glucuronide-MABC linkers, azobenzene linkers, and dialkoxydiphenylsilane linkers.

According to the foregoing, the chemotherapeutic prodrug conjugate according to one embodiment of the present invention may have a form in which the albumin binding moiety is bound to the N-terminus of the peptide linker and, at the same time, the C-terminus of the peptide linker is bound to the anticancer chemotherapeutic agent. Also, conversely, the chemotherapeutic prodrug conjugate according to one embodiment of the invention may have a form in which the albumin binding moiety is bound to the C-terminus of the peptide linker and, at the same time, the N-terminus of the peptide linker is bound to the anticancer chemotherapeutic agent.

According to another embodiment of the invention, the peptide linker in the anticancer chemotherapeutic prodrug conjugate of the present invention is conjugated to the anticancer chemotherapeutic agent via direct or indirect binding of the chemical linker to the albumin binding moiety, and is conjugated to the albumin binding moiety via direct or indirect binding of the peptide linker. For example, dinorubicin exhibits anticancer effects and is conjugated to a functional group such as maleimide or folate at the 14-CH₃ position. Thus, the anticancer effect is achieved through cleavage induced by the caspase and the release of free dinorubicin is not required. Furthermore, according to some embodiments of the present invention, the anticancer chemotherapeutic prodrug conjugate comprises a peptide linker conjugated to daunorubicin via direct or indirect binding of the chemical linker. In this case, the daunorubicin is conjugated to a functional group such as maleimide or folate at the 14-CH3 position via direct or indirect bond of the chemical linker.

### Chemotherapeutic Agent for Cancer

As described above, the prodrug conjugate according to one embodiment of the present invention comprises an anticancer chemotherapeutic agent.

As used herein, the term “anticancer chemotherapeutic agent” refers to a compound (such as a small molecule compound) useful in the treatment of cancer. According to a preferred embodiment, the anticancer chemotherapeutic agent induces cell death within target cells, such as tumor cells and cancer tissue. Any anticancer chemotherapeutic agent known in the art can be used for the prodrug conjugate presented herein.

According to a preferred embodiment, the anticancer chemotherapeutic agent is an anthracycline such as doxorubicin, daunorubicin, epirubicin, idarubicin, valrubicin, or a derivative thereof; an antibiotic such as actinomycin-D, bleomycin, mitomycin-C, calicheamicin, or a derivative thereof; alkylating agent such as cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, duocarmycin, or derivatives thereof; platinum-based agent such as cisplatin, carboplatin, nedaplatin, oxaliplatin, satraplatin, triplatin tetranitrate, or derivatives thereof; antimetabolite such as 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine or derivatives thereof; topoisomerase inhibitor such as camptothecin, topotecan, irinotecan, etoposide, teniposide, mitoxantrone, or derivatives thereof; and mitotic inhibitor such as paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, or a derivative thereof. According to a preferred embodiment, the anticancer chemotherapeutic agent may be doxorubicin or danorubicin.

According to one embodiment, the anticancer chemotherapeutic prodrug conjugate according to the present invention can be provided as a pharmaceutical composition comprising the anticancer chemotherapeutic prodrug conjugate, a pharmaceutically acceptable carrier, additive and/or diluent. Examples of the acceptable carriers, additives, and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, crystalline cellulose, polyvinylpyrrolidone, water, methylhydroxybenzoate, propylhydroxybenzoate, talc, magnesium stearate, minerals, etc.

The pharmaceutical composition according to the present invention can be prepared by any administration method, including parenteral or topical administration. According to some embodiments, the pharmaceutical composition can be preferably injected or administrated intravenously, and may be formulated as a sterile composition for injection or administration. According to other embodiments, the pharmaceutical composition can be formulated as a powder, granule, tablet, capsule, suspension, emulsion, or syrup such that it is suitable for oral administration. According to other embodiments, the pharmaceutical composition can be formulated as a nasal or oral spray or aerosol such that it is suitable for inhalation. According to another embodiment, the pharmaceutical composition can be formulated as a suppository formulation suitable for rectal or intravaginal administration. According to other embodiments, the pharmaceutical composition can be formulated as a solution, emulsion, gel or patch that are suitable for topical or transdermal administration. Suitable compositions and excipients for such compositions can be any of those known in the art.

Examples of solid formulations for oral administration include tablets, pills, powders, granules, capsules, and the like. The solid formulations may be prepared by mixing the conjugate of the present invention with at least one excipient, for example, starch, calcium carbonate, sucrose, lactose, gelatin, etc. In addition to the simple excipients, lubricants such as magnesium stearate or talc may be added during the tableting process or other processes.

Examples of liquid formulations for oral administration include solutions, suspensions, emulsions, and syrups. The liquid formulations may further comprise, for example, water, and optionally liquid paraffin, in addition to various additives such as wetting agents, sweeteners, flavorings, preservatives, and the like.

Examples of formulations for parenteral administration include sterile aqueous solutions, non-aqueous solutions, suspensions, emulsions, lyophilized formulations, and suppositories. Non-aqueous solutions and suspensions include, for example, propylene glycol, polyethylene glycol, vegetable oils such as olive oil, or administerable esters such as ethyl oleate. Base compositions for the suppository formulations include, for example, witepsol, macrogol, tween 61, cacao butter, laurin butter, glycerogelatin, etc.

According to a preferred embodiment, the anticancer chemotherapeutic prodrug conjugate of the present invention may be dissolved in water or in another pharmaceutically acceptable water-soluble carrier, wherein the conjugate optionally has good solubility in other pharmaceutically acceptable excipients, preservatives, etc.

### Method Using Prodrug Conjugates

As described above, the anticancer chemotherapeutic prodrug conjugate according to one embodiment of the present invention are useful in methods of inducing cell death, increasing cell death, and treating cancer. In some embodiments, the anticancer chemotherapeutic prodrug conjugate can be administered to a subject in need of inducing apoptosis of target cells (e.g., tumor cells), increasing apoptosis of the target cells, and/or treating cancer.

In a preferred embodiment, the anticancer chemotherapeutic prodrug conjugate is administerable to an individual in need of treating cancer having KRAS variant or PTEN protein loss genotype.

According to a preferred embodiment, the anticancer chemotherapeutic prodrug conjugate comprising a caspase-cleavable peptide linker according to one embodiment of the present invention can target tumor tissue having KRAS variant or PTEN protein loss genotype, and induce expression of caspase by being taken up into cells and hydrolyzed to cause cell death.

According to some embodiments, those who get general tumor treatment are subjected to a treatment that induces apoptosis in target cells prior to or concurrently with administration of the anticancer chemotherapeutic prodrug conjugate comprising a caspase-cleavable peptide linker according to one embodiment of the present invention, thereby inducing the expression of caspase. According to a preferred embodiment of the invention, the cell death can be induced by any therapeutically acceptable method, which may be selected from the group consisting of radiation therapy, high intensity focused ultrasonic therapy (HIFU), pyrogen therapy, laser therapy, photodynamic therapy, chemotherapeutic therapy and cryosurgery, or targeted therapy such as small molecule tyrosine kinase inhibitors (TKIs) or monoclonal antibodies that target tumor cells. The anticancer chemotherapeutic agent may be any anticancer chemotherapeutic agent known in the art including those mentioned above, and may be the same or different from the anticancer chemotherapeutic agent of the anticancer chemotherapeutic prodrug conjugate. According to a preferred embodiment of the present invention, wherein the tumor or cancer is metastatic or in an unspecified location, the treatment method of inducing cell death comprises targeted treatments that target the tumor cells, such as TKIs, antibodies, aptamers, or targeted nanoparticles.

According to a preferred embodiment, the cell death can be induced by radiation therapy. As used herein, the term “radiation therapy” refers to any method of radiation therapy, including external beam radiation therapy, sealed source radiation therapy, and systemic radioisotope therapy. According to some embodiments, the radiation therapy is locally focused at a target location, such as a tumor location. According to other embodiments, the radiation therapy is effective when preceding the administration of the anticancer chemotherapeutic prodrug conjugate. According to another embodiment using radiation therapy, the radiation therapy comprises gamma knife radiation, cyber knife radiation, and/or high intensity focused ultrasound therapy.

According to some embodiments, the radiation therapy comprises treatment with a low radiation dose. According to a preferred embodiment, an adult subject is treated with a single radiation dose of about 70 Gy. According to another embodiment, an adult subject is treated with a single dose of up to about 35 Gy. According to yet another embodiment, an adult subject is treated with a dose of about 10 Gy per week.

As described above, the anticancer chemotherapeutic prodrug conjugate according to one embodiment of the present invention can be administered via various routes. In a preferred embodiment, the anticancer chemotherapeutic prodrug conjugate can be administered intravenously. The dosage of the anticancer chemotherapeutic prodrug conjugate can be varied depending on the subject and condition to which it is administered and can be determined by one of ordinary skill in the art. In some embodiments, the dosage to a subject may comprise from about mg/kg to about 100 mg/kg, preferably from about 5 mg/kg to about 75 mg/kg, more preferably from about 10 mg/kg to about 50 mg/kg, or more. In more specific embodiments, the anticancer chemotherapeutic prodrug conjugate may have a lower toxicity than the anticancer agent alone, and consequently may have a higher dosage amount than the amount that is non-toxic when the anticancer agent is used alone.

According to some embodiments, the anticancer chemotherapeutic prodrug conjugate according to one embodiment of the present invention may be administered locally to a tumor location, such as topically to a targeted area. According to other embodiments, these targeted areas are areas that have already been treated with the treatment method of inducing cell death as described above.

In some embodiments, the subject is preferably administered an anticancer chemotherapeutic prodrug conjugate followed by further cell death-inducing treatment. In such embodiments, the subsequent cell death-inducing treatment may be the same or equivalent to the previous cell death-inducing treatment. Optionally, the successive cell death-inducing treatments may be different from the previous cell death-inducing treatment. Such differences are not particularly limited by the present invention and may include various types of treatments (e.g., radiation therapy, pyrogen therapy, laser therapy, photodynamic therapy, chemotherapy, cryosurgery, or targeted therapy), chemotherapeutic agent, or targeted molecular therapy. Preferably, radiation therapy can be used, wherein the dose and duration of irradiation can be varied depending on the cell death-inducing treatment.

According to a preferred embodiment, the method of increasing cell death referred to herein comprises the steps below: As mentioned above, the anticancer chemotherapeutic prodrug conjugate is preferentially taken up by tumor tissue having KRAS variant or PTEN protein loss genotype. The caspase cleavable peptide conjugate is hydrolyzed to release the anticancer chemotherapeutic agent and induce cell death, resulting in the expression of caspase. The remaining caspase-cleavable peptide linker of the anticancer chemotherapeutic prodrug conjugate is cleaved by the expressed caspase, further releasing the anticancer chemotherapeutic agent. The anticancer chemotherapeutic agent induces additional cell death, which results in the expression of additional caspases, and therefore triggers additional caspase-induced cleavage activity of the prodrug conjugate and increase of the cell death. This augmentation is a highly efficient and specific method for the killing of target cells (e.g., tumor cells). Furthermore, this augmentation effect has the effect of extending the time between the cell death-inducing treatment and/or the doses of the anticancer chemotherapeutic prodrug conjugate. In some embodiments, the augmentation effect may reduce the amount of anticancer chemotherapeutic agent required to treat a large number of cancer cells.

As mentioned above, the anticancer chemotherapeutic prodrug conjugate according to the present invention are inactive prior to cleavage of the caspase- or cathepsin-cleavable peptide linker. Thus, the anticancer chemotherapeutic prodrug conjugate is non-toxic to (or eliminated in) healthy cells. According to a preferred embodiment, compared to the administration of the same anticancer chemotherapeutic agent in non-conjugated form, the methods presented herein result in about 10%, 20%, 30%, 40%, 50%, 60%, 70%, 80% or more damage reduction in normal cells.

Furthermore, the cell death-inducing effect of the anticancer chemotherapeutic prodrug conjugate with a caspase-cleavable peptide according to the present invention is selective for cells that express the same caspase as the cells undergoing apoptosis. Thus, once the cell death is induced within a target region (e.g., a target tissue), the methods according to the present invention selectively and effectively induce cell death of other target cells, thereby treating cancer, for examples.

The cell death-inducing effect of the anticancer chemotherapeutic prodrug conjugate with a cathepsin-cleavable peptide according to one embodiment of the present invention is selective for cells in which the cathepsin enzyme is overexpressed. Thus, when the cathepsin enzyme is overexpressed in a majority of tumor cells, the method according to the present invention treats cancer by selectively and effectively killing only the tumor cells in which the enzyme is overexpressed.

The cell death-inducing effect of the anticancer chemotherapeutic prodrug conjugate according to the present invention is selective and effective against tumor tissue having KRAS variants or PTEN protein loss genotypes, thereby treating cancer.

### MODE FOR PRACTICING THE INVENTION

The present invention will now be described in more detail with reference to the following examples. However, the present invention is not limited to the embodiments disclosed herein, but may be embodied in many different forms. The following embodiments are provided to make the disclosure of the invention complete and to give those of ordinary skill in the art a complete idea of the scope of the invention.

### Example 1: Preparation of Maleimide-KGDEVD-PABC-Doxorubicin Prodrug Conjugate

A prodrug having the structure of Scheme 1 below, where a maleimide group that can bind to thiol group has been derived at the N-terminus of a Lys-Gly-Asp-Glu-Val-Asp peptide (SEQ ID NO: 32) and particularly doxorubicin as an active ingredient of an anticancer agent is chemically linked at the C-terminus, was prepared by the method shown in Reaction 1 below.

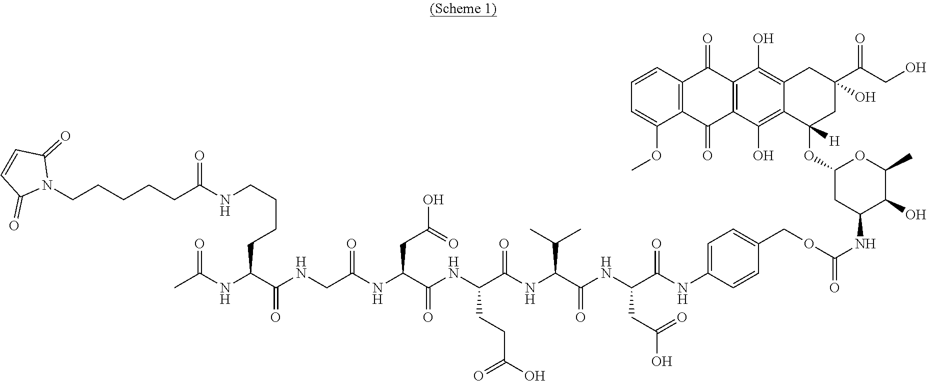 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(=O)O)NC(=O)CNC(=O)[C@H](CCCCNC(=O)CCCCCN3C(=O)C=CC3=O)NC(C)=O)C(C)C)cc2)[C@H](O)[C@H](C)O1]

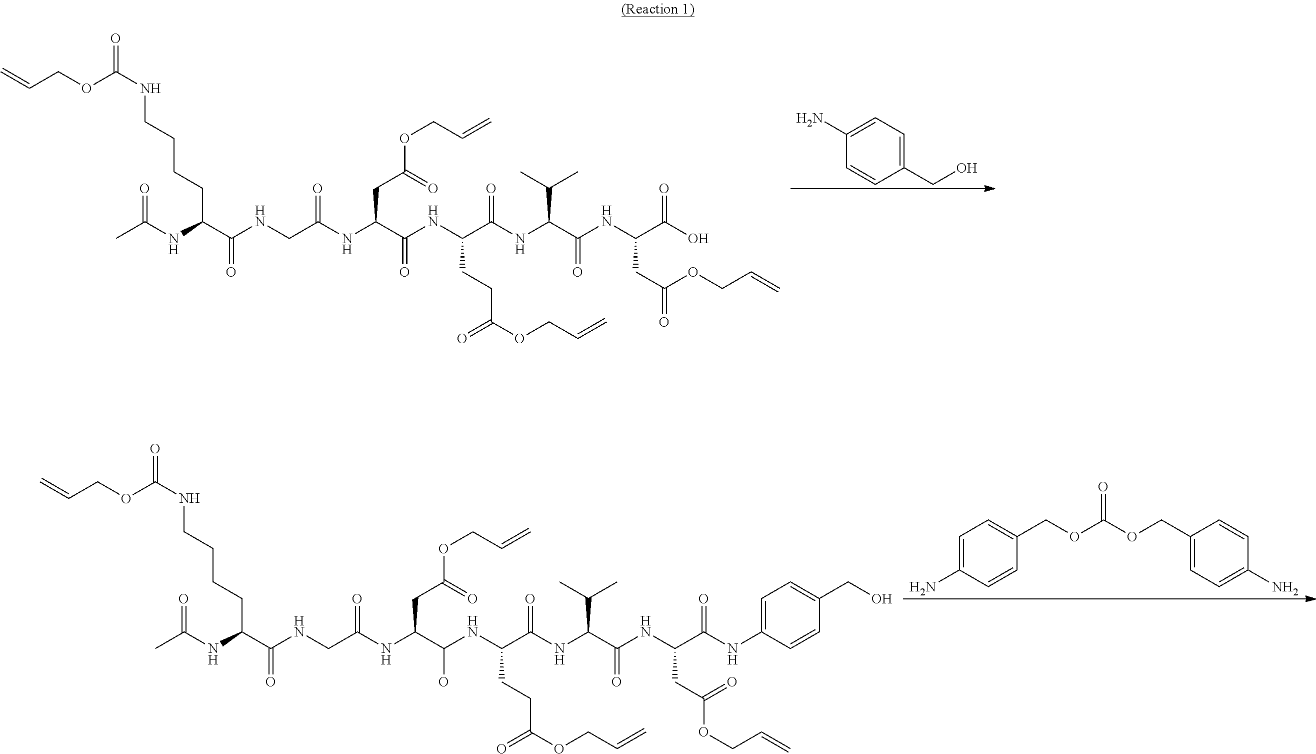

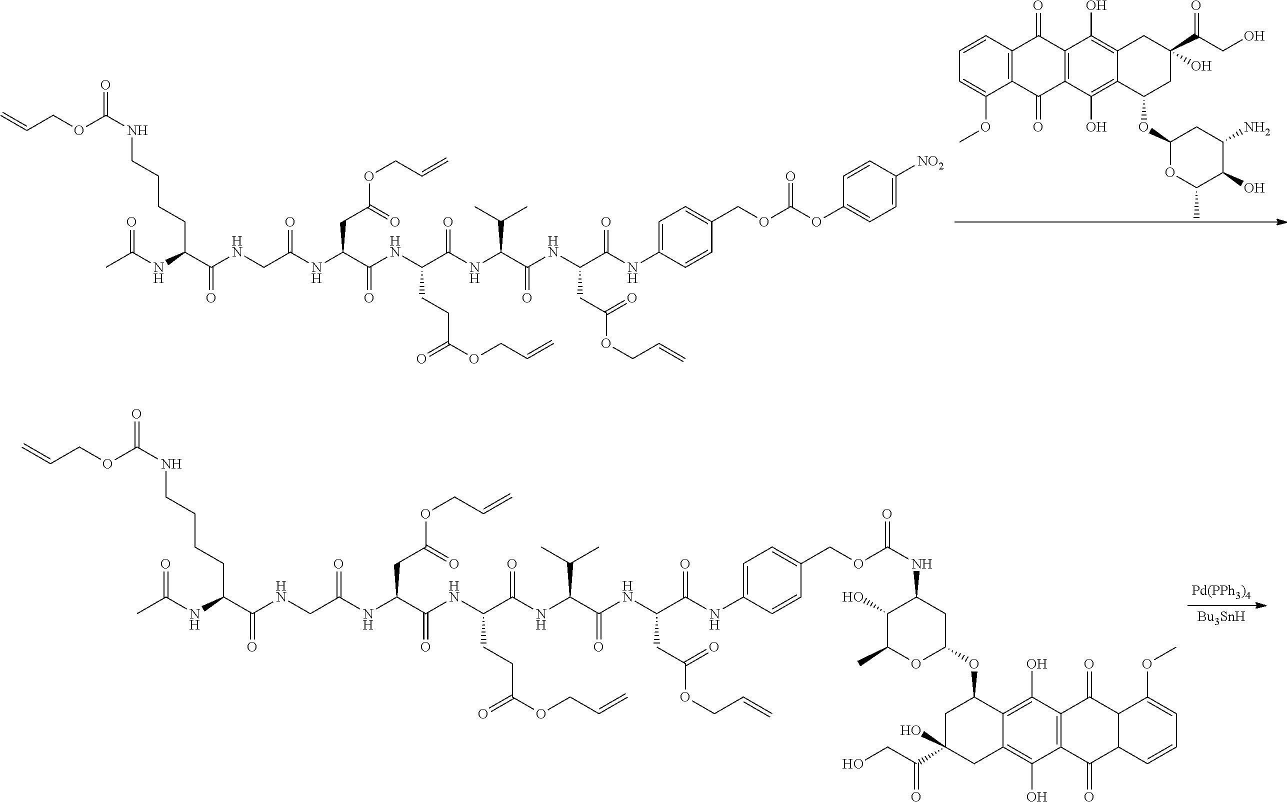

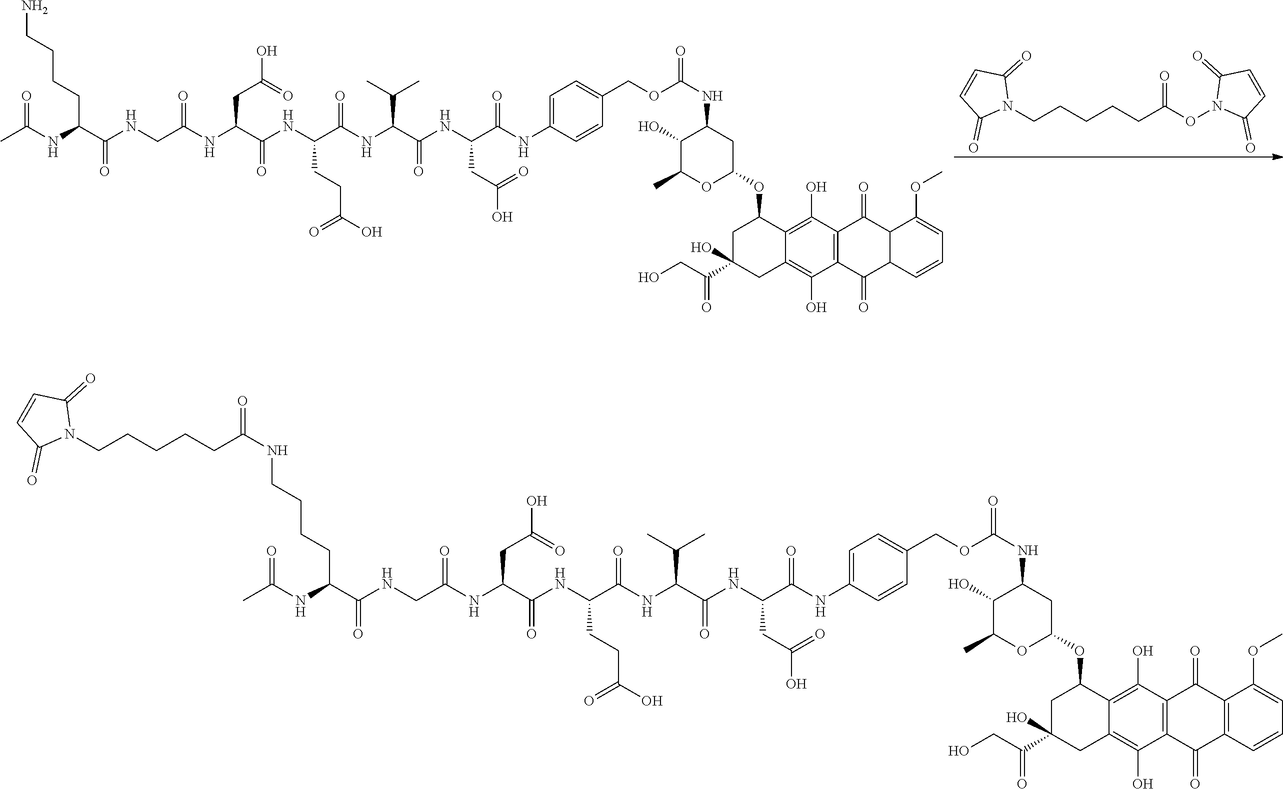

The substance disclosed in this embodiment has a maleimide group that binds to the thiol group of endogenous albumin after intravenous administration, and a linker comprising a Lys-Gly-Asp-Glu-Val-Asp peptide (SEQ ID NO: 32) that is enzymatically hydrolyzed by caspase-3 which is activated in irradiated tumors, linked to the anticancer chemotherapeutic agent doxorubicin. The endogenous albumin mediates delivery of the substance to tumor tissue which is followed by the release of doxorubicin by the caspase-3.

More specifically describing the preparation of the prodrug conjugate of the present invention, firstly, Ac-Lys(OAloc)-Gly-Asp(OAll)-Glu(OAll)-Val-Asp(OAll)-OH (1000 mg, 1.10 mmol), 4-aminobenzyl alcohol (2 eq) and 2-ethoxy-2H-quinoline-1-carboxylic acid ethyl ester (EEDQ, 2 eq) were dissolved in anhydrous dimethylformamide (DMF, 40 mL) and stirred at room temperature under nitrogen gas for 16 h. The solution was concentrated in vacuo and crystallized using ether to give Ac-Lys(OAloc)-Gly-Asp(OAll)-Glu(OAll)-Val-Asp(OAll)-PABOH.

The solution of the above Ac-Lys(OAloc)-Gly-Asp(OAll)-Glu(OAll)-Val-Asp(OAll)-PABOH (1,095 mg, 1.08 mmol) and bis(p-nitrophenyl)carbonate (5.4 eq) were dissolved in anhydrous DMF (35 mL) and stirred at room temperature under nitrogen gas. N,N-diisopropylethylamine (DIPEA, 3.2 eq) was then added slowly and stirred for 16 h. The solution was concentrated in vacuo and crystallized using ether to give Ac-Lys(OAloc)-Gly-Asp(OAll)-Glu(OAll)-Val-Asp(OAll)-PABC.

The solution of the above Ac-Lys(OAloc)-Gly-Asp(OAll)-Glu(OAll)-Val-Asp(OAll)-PABC (998 mg, 0.9 mmol) and doxorubicin hydrochloride (1.2 eq) were dissolved in anhydrous DMF (35 mL) and stirred under nitrogen gas in a dark environment. DIPEA (5.4 eq) was then added slowly and stirred at room temperature for 12 h. The solution was concentrated in vacuo and precipitated from water to give Ac-Lys(OAloc)-Gly-Asp(OAll)-Glu(OAll)-Val-Asp(OAll)-PABC-Dox.

The above Ac-Lys(OAloc)-Gly-Asp(OAll)-Glu(OAll)-Val-Asp(OAll)-PABC-Dox (1,240 mg, 0.8 mmol) was dissolved in a solution of chloroform/acetic acid/4-methylmorpholine in a ratio of 37/2/1 (510 mL in total), and the above mixture was stirred under nitrogen gas. Tetrakis(triphenylphosphine)palladium {Pd(PPh₃)₄, 3 eq} was then dissolved in chloroform (21 mL) and slowly added to the above mixed solution and stirred at room temperature for 6 h. The precipitate formed after the end of the reaction was obtained by separating it from the mixture, dried, and then dissolved in water. Subsequently, it was purified by semi-preparative HPLC using a C18 reverse-phase column (water/CH₃CN, 1% acetic acid as an additive, CH₃CN 20-100% over 50 min, 5 ml/min) to give deprotected Ac-Lys-Gly-Asp-Glu-Val-Asp-PABC-DOX as a red amorphous solid. ESI-MS: m/z 1378.4 [M+H]⁺.

The above Ac-Lys-Gly-Asp-Glu-Val-Asp-PABC-DOX and N-(ε-Maleimido-caproxyloxy)succinimide ester (EMCS, 2 eq) were dissolved in anhydrous DMF followed by addition of triethylamine (2.5 eq) and stirring at room temperature for 2 h.

The final product was separated by semi-preparative HPLC (Shimadzu, Kyoto, Japan) of a concentration gradient system (water/CH₃CN, 0.05% trifluoroacetic acid (TFA) as an additive, CH₃CN 20-50% over 50 min, 8 ml/min) using an ODS-A 5 μm reversed semi-preparative column (150 mm×20 mm), which yielded Ac-Lys(EMC)-Gly-Asp-Glu-Val-Asp-PABC-DOX as a red amorphous solid. (The EMC stands for ε-maleimidocapric acid).

The peaks were monitored at 290 nm. The separation of the final product was confirmed by analytical HPLC (Agilent 1300 series, Agilent Technologies, Santa Clara, CA) of a concentration gradient system (water/CH₃CN, 0.1% TFA as an additive, CH₃CN 20-50% over 50 min, 8 ml/min). The peaks were monitored by UV detector (214 nm) and fluorescence detector (excitation 470 nm, emission 580 nm). The purity of the final product was found to be greater than 95%. ESI-MS (m/z): 1593.3 [M+Na]⁺.

### Example 2: Preparation of Maleimide-KGDEVD-PABC-Daunorubicin Prodrug Conjugate

A maleimide-KGDEVD-PABC-daunorubicin prodrug conjugate having the structure of Scheme 2 below was prepared in the same manner as in Example 1 except that daunorubicin was used instead of doxorubicin. (KGDVED corresponds to SEQ ID NO: 32)

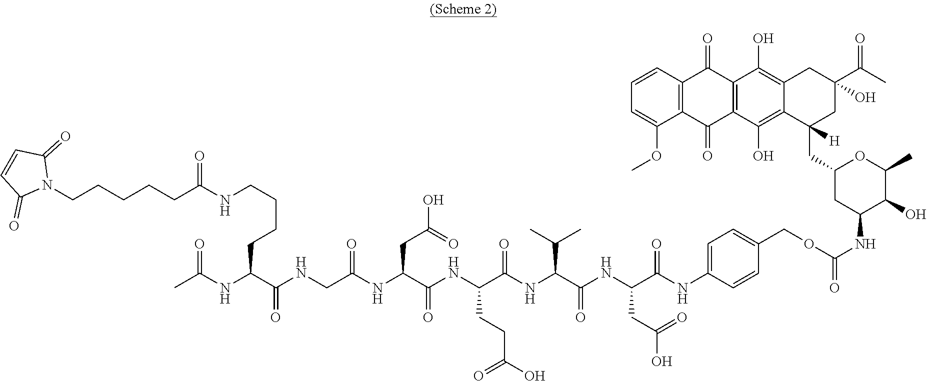 [SMILES: COc1cccc2c1C(=O)c1c(O)c3c(c(O)c1C2=O)C[C@@](O)(C(C)=O)C[C@@H]3C[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(=O)O)NC(=O)CNC(=O)[C@H](CCCCNC(=O)CCCCCN3C(=O)C=CC3=O)NC(C)=O)C(C)C)cc2)[C@H](O)[C@H](C)O1]

### Example 3: Preparation of Maleimide-KGDEVD-PABC-Paclitaxel Prodrug Conjugate

A maleimide-KGDEVD-PABC-paclitaxel prodrug conjugate having the structure of Scheme 3 below was prepared in the same manner as in Example 1 except that paclitaxel was used instead of doxorubicin. (KGDVED corresponds to SEQ ID NO: 32)

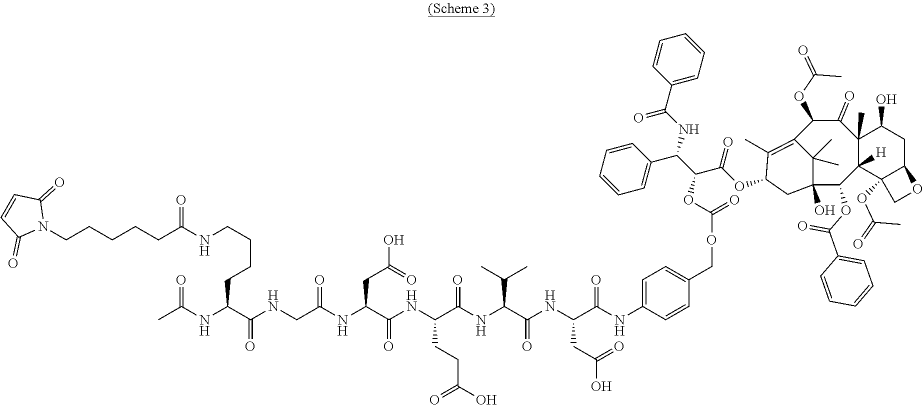

### Example 4: Preparation of Maleimide-KGDEVD-PABC-MMAE Prodrug Conjugate

A maleimide-KGDEVD-MMAE prodrug conjugate having the structure of Scheme 4 below was prepared in the same manner as in Example 1 except that monomethyl auristatin E (MMAE) was used instead of doxorubicin. (KGDVED corresponds to SEQ ID NO: 32)

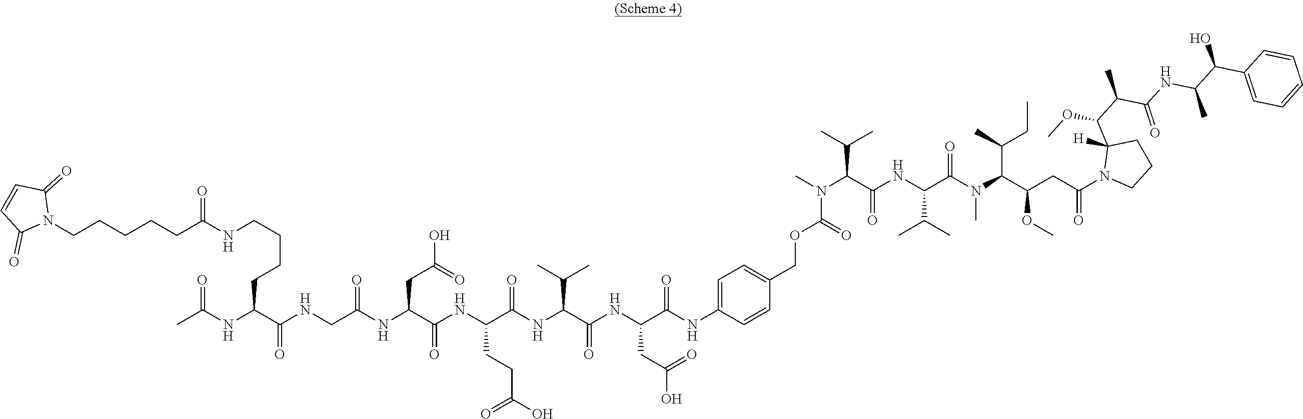 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(=O)O)NC(=O)[C@@H](NC(=O)[C@H](CCC(=O)O)NC(=O)[C@H](CC(=O)O)NC(=O)CNC(=O)[C@H](CCCCNC(=O)CCCCCN2C(=O)C=CC2=O)NC(C)=O)C(C)C)cc1)C(C)C]

### Example 5: Preparation of Maleimide-DEVD-PABC-Doxorubicin Prodrug Conjugate

A maleimide-DEVD-doxorubicin prodrug conjugate having the structure of Scheme 5 below was prepared in the same manner as in Example 1, except that DEVD (SEQ ID NO: 13) was synthesized instead of the peptide linker KGDEVD (SEQ ID NO: 32). DEVD can be cleaved by caspase-3, caspase-7, and caspase-9.

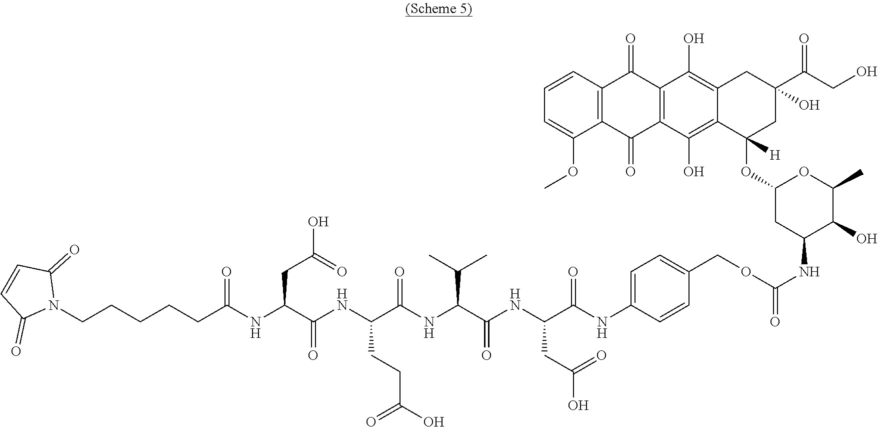 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(=O)O)NC(=O)CCCCCN3C(=O)C=CC3=O)C(C)C)cc2)[C@H](O)[C@H](C)O1]

### Example 6: Preparation of Maleimide-DEID-PABC-Doxorubicin Prodrug Conjugate

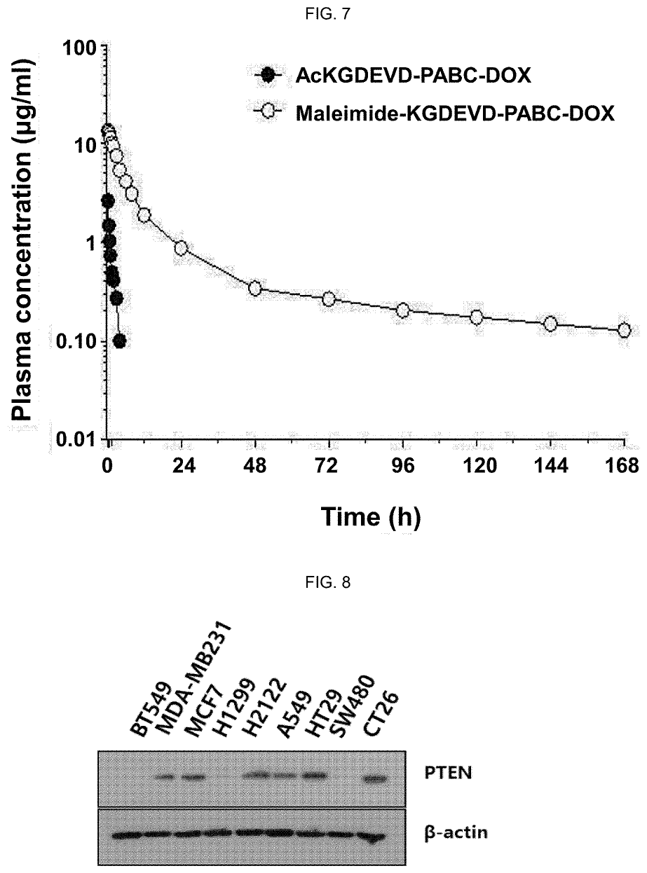

A maleimide-DEID-doxorubicin prodrug conjugate having the structure of Scheme 6 below was prepared in the same manner as in Example 1 except that the peptide linker DEID (SEQ ID NO: 15) cleavable by caspase was synthesized instead of the peptide linker KGDEVD (SEQ ID NO: 32) (FIG. 7). The DEID peptide linker can be cleaved by casepase-3, casepase-7, and casepase-9.

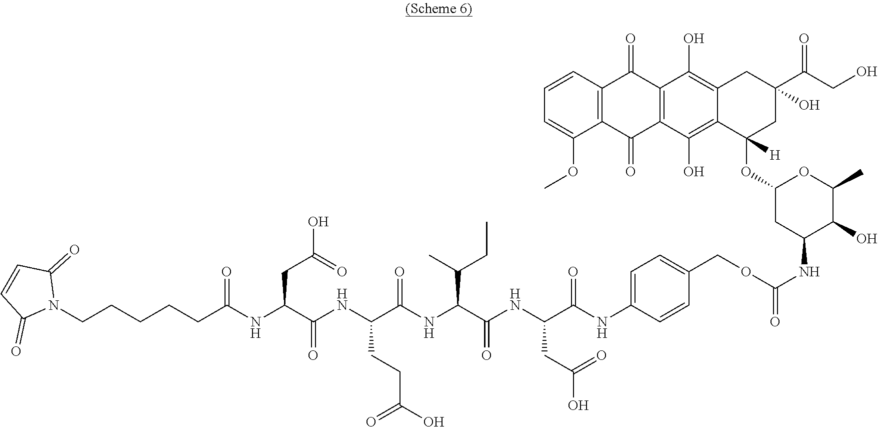 [SMILES: CCC(C)[C@H](NC(=O)[C@H](CCC(=O)O)NC(=O)[C@H](CC(=O)O)NC(=O)CCCCCN1C(=O)C=CC1=O)C(=O)N[C@@H](CC(=O)O)C(=O)Nc1ccc(COC(=O)N[C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)cc1]

### Example 7: Preparation of Maleimide-DLVD-PABC-Doxorubicin Prodrug Conjugate

A maleimide-DLVD-doxorubicin prodrug conjugate having the structure of Scheme 7 below was prepared in the same manner as in Example 1, except that the peptide linker DLVD (SEQ ID NO: 14) cleavable by caspase was synthesized instead of the peptide linker KGDEVD (SEQ ID NO: 32). The DLVD can be cleaved by caspase-3, caspase-7, and caspase-9.

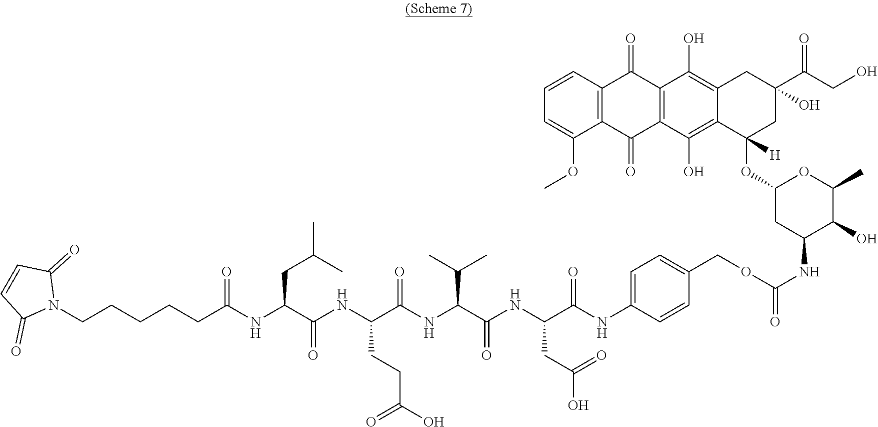 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(C)C)NC(=O)CCCCCN3C(=O)C=CC3=O)C(C)C)cc2)[C@H](O)[C@H](C)O1]

### Example 8: Preparation of Maleimide-DEVD-Doxorubicin (without PABC) Prodrug Conjugate

A maleimide-DEVD-doxorubicin prodrug conjugate having the structure of Scheme 8 below was synthesized using the same method as in Example 5 except for the process of linking the PABC linker. In this embodiment, the prodrug conjugate maleimide-DEVD-doxorubicin was prepared by directly connecting the DEVD peptide linker to the 3′-NH₂ of doxorubicin. (DEVD corresponds to SEQ ID NO: 13)

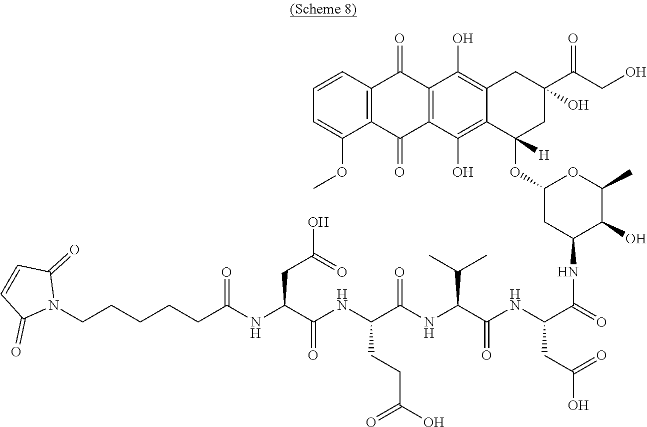 [SMILES: COc1cccc2c1C(=O)c1c(O)c3c(c(O)c1C2=O)C[C@@](O)(C(=O)CO)C[C@@H]3O[C@H]1C[C@H](NC(=O)[C@H](CC(=O)O)NC(=O)[C@@H](NC(=O)[C@H](CCC(=O)O)NC(=O)[C@H](CC(=O)O)NC(=O)CCCCCN2C(=O)C=CC2=O)C(C)C)[C@H](O)[C@H](C)O1]

### Example 9: Preparation of Maleimide-DEVD-MMAE (without PABC) Prodrug Conjugate

A maleimide-DEVD-MMAE having the structure of Scheme 9 below was prepared by the same method as in Example 8 except that MMAE was used instead of doxorubicin as the anticancer chemotherapeutic agent. (DEVD corresponds to SEQ ID NO: 13)

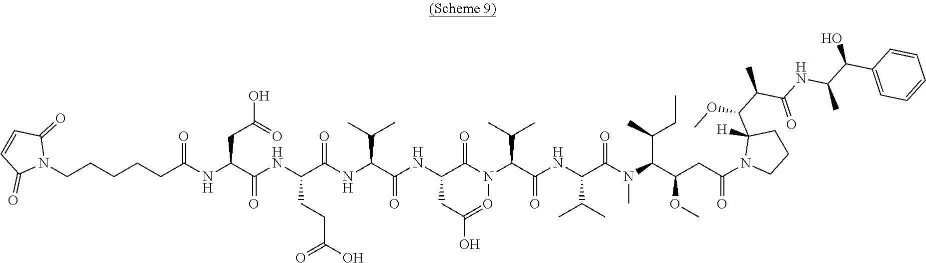

### Example 10: Preparation of Pyridyldithiol-KGDEVD-PABC-Doxorubicin Prodrug Conjugate

A pyridyldithiol-doxorubicin prodrug conjugate having the structure of Scheme 10 below was prepared using the same method as in Example 1 except that a pyridyldithiol group was used instead of maleimide. The pyridyldithiol functional group binds through the endogenous albumin-disulfide bonding and prolongs the prodrug conjugate plasma circulation time. In this embodiment, 3-(2-pyridyldithiol)propionate and doxorubicin are conjugated to the N- and C-termini of KGDEVD (SEQ ID NO: 32) that can be cleaved by caspase-3, caspase-7, and caspase-9, respectively. As with other prodrug conjugates exemplified above, this prodrug conjugate covalently binds to circulating endogenous albumin and accumulates in tumor cells. When cell death is induced and caspase-3 or caspase-7 is activated, the caspase-cleavable peptide linker is cleaved and releases doxorubicin.

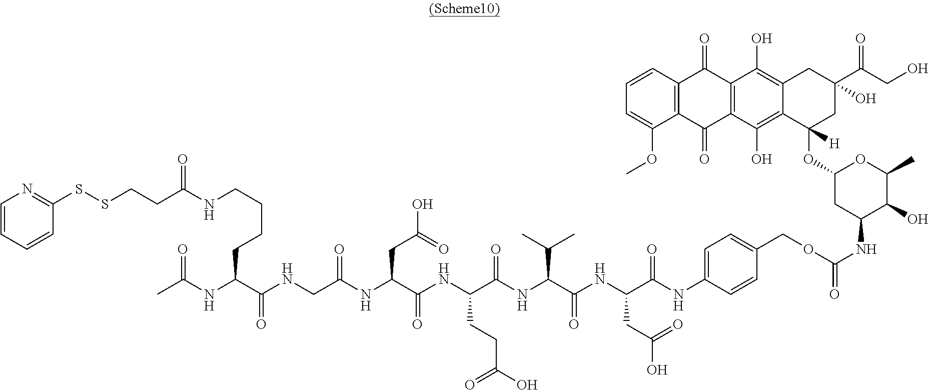 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(=O)O)NC(=O)CNC(=O)[C@H](CCCCNC(=O)CCSSc3ccccn3)NC(C)=O)C(C)C)cc2)[C@H](O)[C@H](C)O1]

### Example 11: Preparation of Oleate-KGDEVD-PABC-Doxorubicin Prodrug Conjugate

A prodrug conjugate similar to Example 10, in particular the oleate-KGDEVD-PABC-doxorubicin prodrug conjugate comprising oleate as an albumin binding moiety was prepared, whose structure is illustrated in Scheme 11 below. The oleate functional group binds to the endogenous albumin and prolongs the plasma circulation time of the prodrug conjugate. (KGDVED corresponds to SEQ ID NO: 32)

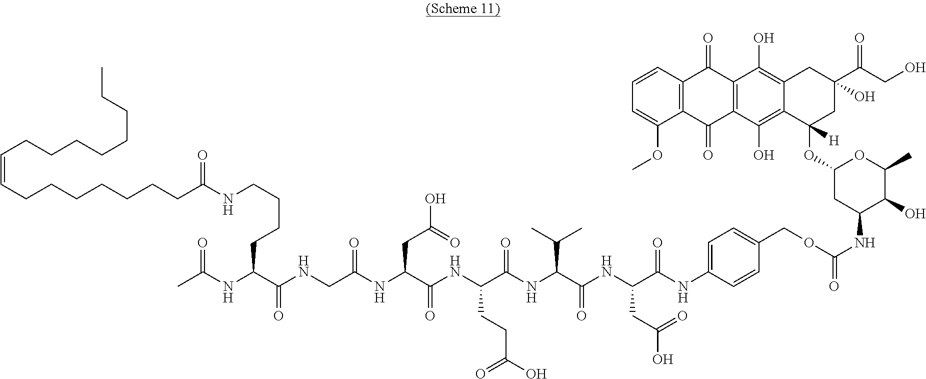 [SMILES: CCCCCCCC/C=C\CCCCCCCC(=O)NCCCC[C@H](NC(C)=O)C(=O)NCC(=O)N[C@@H](CC(=O)O)C(=O)N[C@@H](CCC(=O)O)C(=O)N[C@H](C(=O)N[C@@H](CC(=O)O)C(=O)Nc1ccc(COC(=O)N[C@H]2C[C@H](O[C@H]3C[C@](O)(C(=O)CO)Cc4c(O)c5c(c(O)c43)C(=O)c3c(OC)cccc3C5=O)O[C@@H](C)[C@H]2O)cc1)C(C)C]

### Example 12: Preparation of Folate-KGDEVD-PABC-Doxorubicin Prodrug Conjugate

A prodrug conjugate similar to Example 10, in particular the folate-KGDEVD-PABC-doxorubicin prodrug conjugate comprising folate as an albumin binding moiety was prepared, whose structure is illustrated in Scheme 14 below. (KGDEVD corresponds to SEQ ID NO: 32)

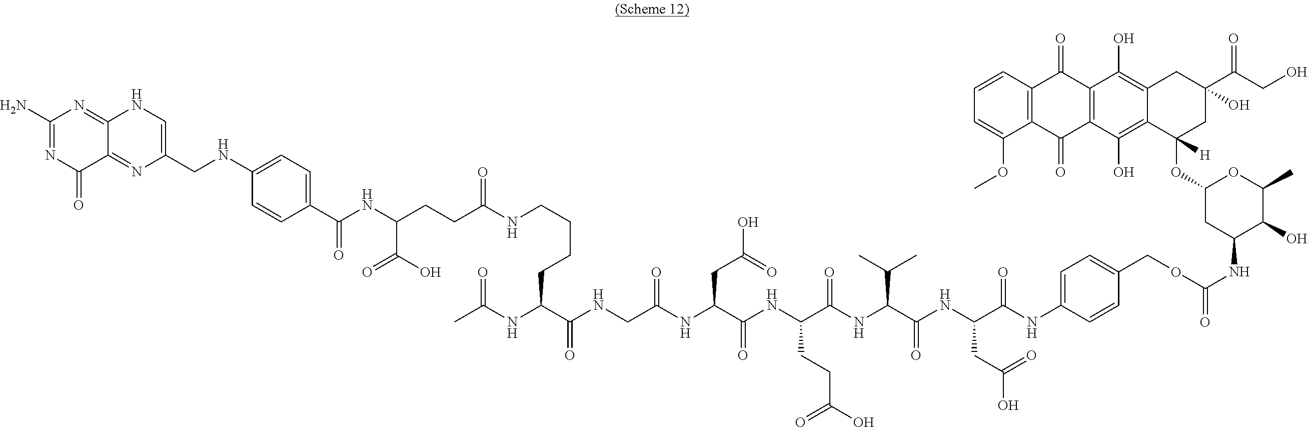 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CC(=O)O)NC(=O)[C@@H](NC(=O)[C@H](CCC(=O)O)NC(=O)[C@H](CC(=O)O)NC(=O)CNC(=O)[C@H](CCCCNC(=O)CCC(NC(=O)c3ccc(NCc4c[nH]c5nc(N)nc(=O)c-5n4)cc3)C(=O)O)NC(C)=O)C(C)C)cc2)[C@H](O)[C@H](C)O1]

### Example 13: Preparation of Maleimide-KGRRG-PABC-Doxorubicin Prodrug Conjugate

A maleimide-KGRRG-doxorubicin prodrug conjugate having the structure of Scheme 20 below was prepared in the same manner as in Example 1, except that the peptide linker KGRRG (SEQ ID NO: 40) cleavable by cathepsin D was synthesized instead of the peptide linker KGDEVD (SEQ ID NO: 32). The KGRRG can be cleaved by cathepsin D.

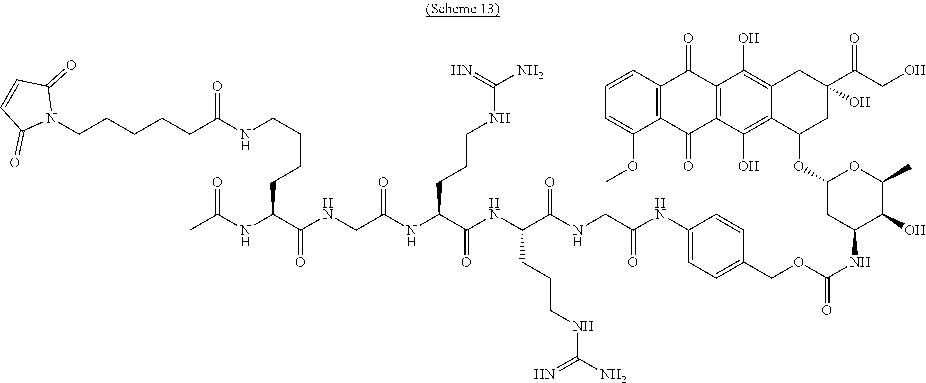 [SMILES: COc1cccc2c1C(=O)c1c(O)c3c(c(O)c1C2=O)C[C@@](O)(C(=O)CO)CC3O[C@H]1C[C@H](NC(=O)OCc2ccc(NC(=O)CNC(=O)[C@H](CCCNC(=N)N)NC(=O)[C@H](CCCNC(=N)N)NC(=O)CNC(=O)[C@H](CCCCNC(=O)CCCCCN3C(=O)C=CC3=O)NC(C)=O)cc2)[C@H](O)[C@H](C)O1]

### Experimental Example 1: Conjugate Binding of Maleimide-KGDEVD-PABC-Doxorubicin with Human Serum Albumin and its Analysis Through HPLC

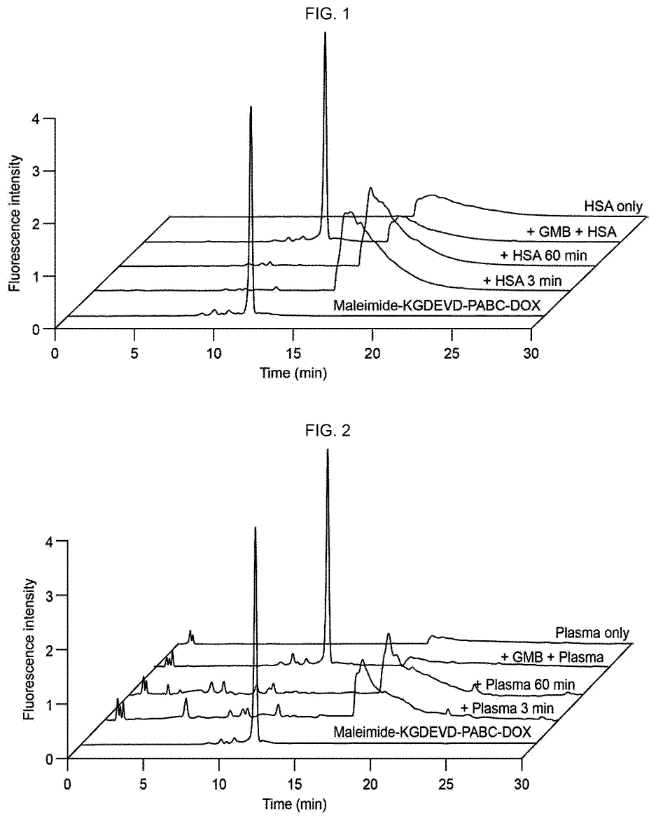

Commercially available human serum albumin (HSA, Sigma-Aldrich, USA) was dissolved in PBS to a concentration of 700 μM. The maleimide-KGDEVD-PABC-doxorubicin synthesized in Example 1 was then added to a concentration of 100 μM and incubated at room temperature. The samples were analyzed by analytical HPLC (FIG. 1). (KGDEVD corresponds to SEQ ID NO: 32)

Conjugate binding of maleimide-KGDEVD-PABC-doxorubicin with HSA was complete in 3 min and a small amount of unbound material was detected. However, when HSA was pre-incubated with 4-maleimidobutyric acid which has a thiol-bound maleimide group, prior to incubation with maleimide-KGDEVD-PABC-doxorubicin, no binding of maleimide-KGDEVD-PABC-doxorubicin to HSA was observed even after 1 h. This suggests that the binding of maleimide-KGDEVD-PABC-doxorubicin is specifically mediated through the maleimide functional group of the EMC functional group.

### Experimental Example 2: Conjugate Binding of Maleimide-KGDEVD-PABC-Doxorubicin with Human Serum Albumin in Plasma and its Analysis Through HPLC

To investigate whether the prodrug conjugate according to one embodiment of the present invention can also bind to serum albumin in human blood upon administration, the present inventors added the maleimide-KGDEVD-PABC-doxorubicin synthesized in Example 1 (KGDEVD corresponds to SEQ ID NO: 32) to a human plasma sample to a concentration of 100 μM and incubated at room temperature, after which the sample was analyzed by analytical HPLC (FIG. 2). The conjugation binding of maleimide-KGDEVD-PABC-doxorubicin with HSA was completed in 3 min. However, when plasma was pre-incubated with 4-maleimidobutyric acid which has a thiol-bound maleimide functional group, prior to incubation with maleimide-KGDEVD-PABC-doxorubicin no, binding of maleimide-KGDEVD-PABC-doxorubicin to plasma was observed even after 1 h as in the previous experiment conducted with serum. This suggests that the binding of maleimide-KGDEVD-PABC-doxorubicin is specifically mediated through the maleimide functional group of the EMC functional group.

### Experimental Example 3: Verifying the Release of from Maleimide-KGDEVD-PABC-Doxorubicin (HSA-KGDEVD-PABC-Doxorubicin) by Caspase-3 Using HPLC Analysis

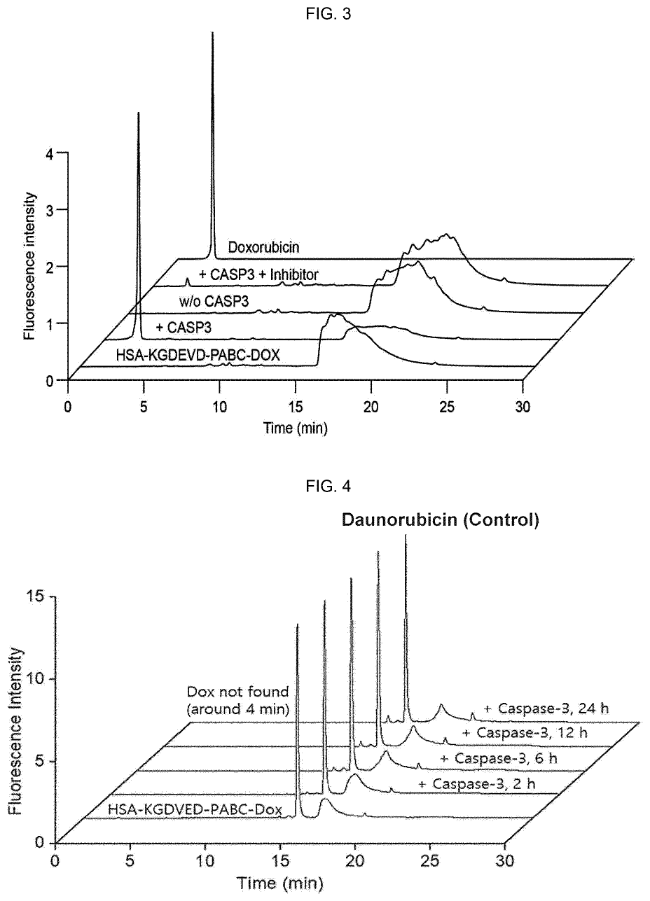

The HSA-Maleimide-KGDEVD-PABC-doxorubicin was incubated with purified caspase-3 and analyzed by HPLC (FIG. 3). (KGDEVD corresponds to SEQ ID NO: 32) Doxorubicin was cleaved and isolated from the HSA-maleimide-KGDEVD-PABC-doxorubicin conjugate within 1 h. In contrast, no cleavage was observed for the HSA-Maleimide-KGDVED-PABC-doxorubicin conjugate with a different peptide amino acid sequence (FIG. 4).

### Experimental Example 4: In Vitro Anticancer Effect of HSA-KGDEVD-PABC-Doxorubicin at Different Concentrations in the Presence and Absence of Human Caspase-3

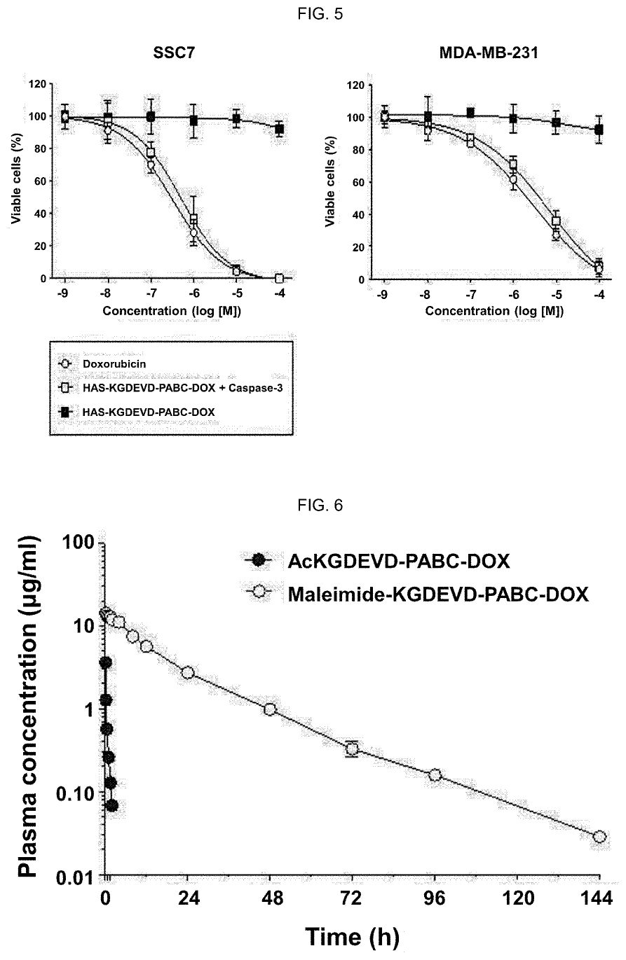

The HSA-maleimide-KGDEVD-PABC-doxorubicin conjugate (KGDEVD corresponds to SEQ ID NO: 32) did not show any obvious toxic effects in SCC7 and MDA-MB-231 cells at concentrations up to 100 μM when tested by MTT analysis. However, when the HSA-maleimide-KGDEVD-PABC-doxorubicin was pre-incubated with purified caspase-3 prior to cell addition, it exhibited a similar degree of cytotoxicity as the doxorubicin without covalent bonding in SCC7 and MDAMB-231 (FIG. 5).

### Experimental Example 5: Pharmacokinetic Analysis in Rats

The present inventors analyzed the pharmacokinetic profile of the maleimide-KGDEVD-PABC-doxorubicin and AcKGDEVD-PABC-doxorubicin conjugates synthesized in Example 1 (KGDEVD corresponds to SEQ ID NO: 32) after intravenous administration to Sprague-Dawley rats at an amount equivalent to 1 mg/kg based on doxorubicin (FIG. 6). Blood samples were collected at 5, 15, 30, 60, and 90 min intervals and then again at 2, 4, 8, 12, 24, 48, 72, 96, and 144 h. The blood samples were stabilized with sodium citrate and then cryocentrifuged at 2000×g for 15 min to obtain plasma. 200 μl of the plasma sample was placed in a 96-well black microplate and the fluorescence of doxorubicin was measured at 485/590 nm. The reference was fresh plasma and the fluorescence was measured in the same manner.

The results showed that the maleimide-KGDEVD-PABC-doxorubicin had a terminal half-life of 30 min and the plasma concentrations decreased below the detection limit of 5 ng/ml within 4 h. In contrast, the maleimide-KGDEVD-PABC-doxorubicin exhibited an extended half-life of more than 19 h and remained active in plasma for 6 days after administration.

### Experimental Example 6: Pharmacokinetic Analysis in Cynomolgus Monkeys

The present inventors analyzed the pharmacokinetic profile of the maleimide-KGDEVD-PABC-doxorubicin and AcKGDEVD-PABC-doxorubicin conjugates synthesized in Example 1 (KGDEVD corresponds to SEQ ID NO: 32) after intravenous administration to cynomolgus monkeys at an amount equivalent to 1 mg/kg based on doxorubicin (FIG. 7). Blood samples were collected at 15, 30, 45, 60, and 90 min intervals, and then again at 2, 3, 4, 6, 8, 12, 24, 48, 72, 96, 120, 144, and 168 h. The blood samples were stabilized with sodium citrate and then cryocentrifuged at 2000×g for 15 min to obtain plasma. 200 μl of the plasma sample was added to a 96-well black microplate and the fluorescence of doxorubicin was measured at 485/590 nm. The reference was fresh plasma and the fluorescence was measured in the same manner. The AcKGDEVD-PABC-doxorubicin showed a terminal half-life of 1 h, and the plasma concentration decreased below the detection limit of 5 ng/ml within 6 h. In contrast, the maleimide-KGDEVD-PABC-doxorubicin exhibited an extended half-life of more than 106 h and was detectable in plasma for 7 days after administration.

### Experimental Example 7: Western Blot Evaluation to Identify Cell Populations with PTEN Protein Loss Genotype

The present inventors performed Western blot analysis in various cell lines (breast cancer: BT549, MDA-MB231, MCF7; lung cancer: H1299, H2122, A549; colon cancer: HT29, SW480, CT26) to identify cell lines with PTEN protein loss genotype. The results showed that PTEN protein was not detected in BT549 breast cancer cells, H1299 lung cancer cells, and SW480 colon cancer cells, confirming that the PTEN protein loss genotype was expressed in those cell lines (FIG. 8).

### Experimental Example 8: Evaluation of Intracellular Uptake of Human Serum Albumin Into Tumor Cells with KRAS Variant Genotype and with Wild-Type KRAS Genotype

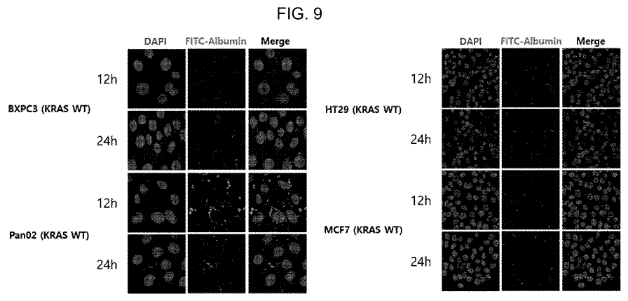

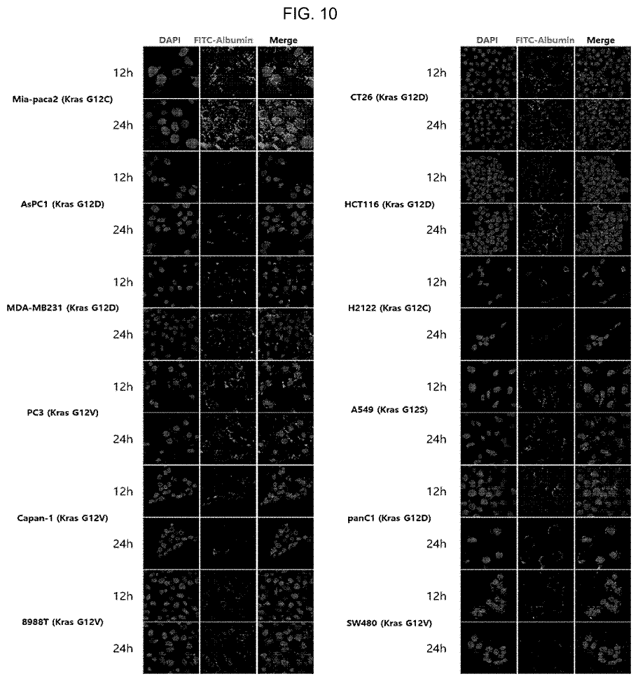

When treating the human serum albumin to tumor cell lines with wild-type KRAS genotype (pancreatic cancer: BXPC3, Pan02; prostate cancer: DU145; colorectal cancer: HT29; breast cancer: MCF7) and those with KRAS variant genotype (pancreatic cancer: AsPC3, Mia-paca2, Capan-1, panC1, 8988T; prostate cancer: PC3; colorectal cancer: CT26, HCT116, SW480; lung cancer: H2122, A549), the tumor cell lines with KRAS variant genotype showed large amounts of intracellular albumin uptake. In contrast, it was not found in the tumor cell lines with wild-type KRAS genotype (FIG. 9 and FIG. 10).

### Experimental Example 9: Evaluation of Intracellular Uptake of Maleimide-KGDEVD-Doxorubicin into Tumor Cells with KRAS Variant Genotype and Wild-Type KRAS Genotype

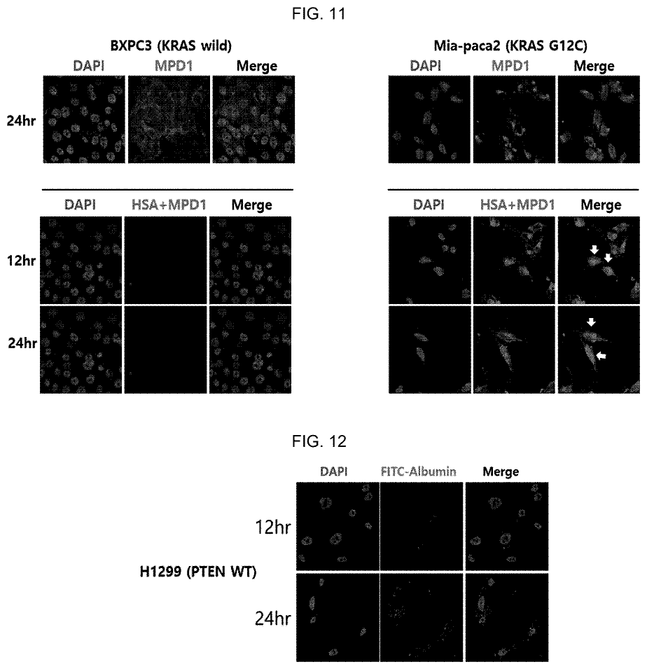

When tumor cell lines with wild-type KRAS genotype (pancreatic cancer: BXPC3) and those with KRAS variant genotype (pancreatic cancer: Mia-paca2) were treated with the albumin-binding prodrug conjugate, where the maleimide-KGDEVD-PABC-doxorubicin conjugate synthesized in Example 1 (KGDEVD corresponds to SEQ ID NO: 32) is bound to the human serum albumin, large amounts of uptake of the albumin-binding prodrug conjugate was found in the tumor cells with KRAS variant genotype (FIG. 11).

### Experimental Example 10: Evaluation of Intracellular Uptake of Human Serum Albumin into Tumor Cells with PTEN Protein Loss Genotype and Wild-Type PTEN Protein Genotype

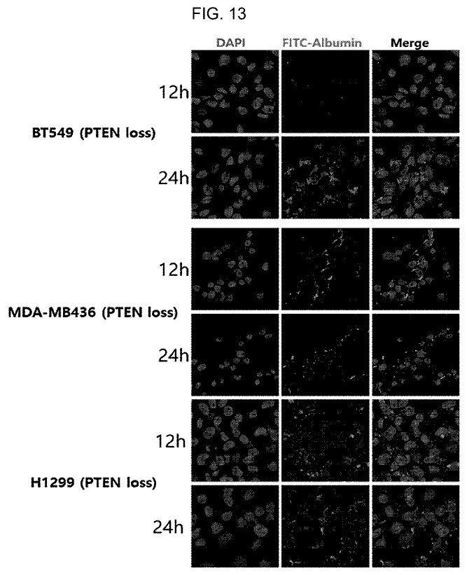

When tumor cell lines with wild-type PTEN protein genotype (prostate cancer: DU145; colorectal cancer: HT29) and those with PTEN protein loss genotype (breast cancer: BT579, MDA-MB436; lung cancer: H1299) were treated with human serum albumin fluorescently labeled with FITC, large amounts of intracellular albumin uptake in the tumor cell lines with PTEN protein loss genotype was found. In contrast, it was not found in those with wild-type PTEN protein genotype (FIG. 12 and FIG. 13).

### Experimental Example 11: Evaluation of Cytotoxicity at Different Concentrations of Maleimide-KGDEVD-PABC-Doxorubicin in Tumor Cells with KRAS Variant Genotype and Normal Tumor Cells

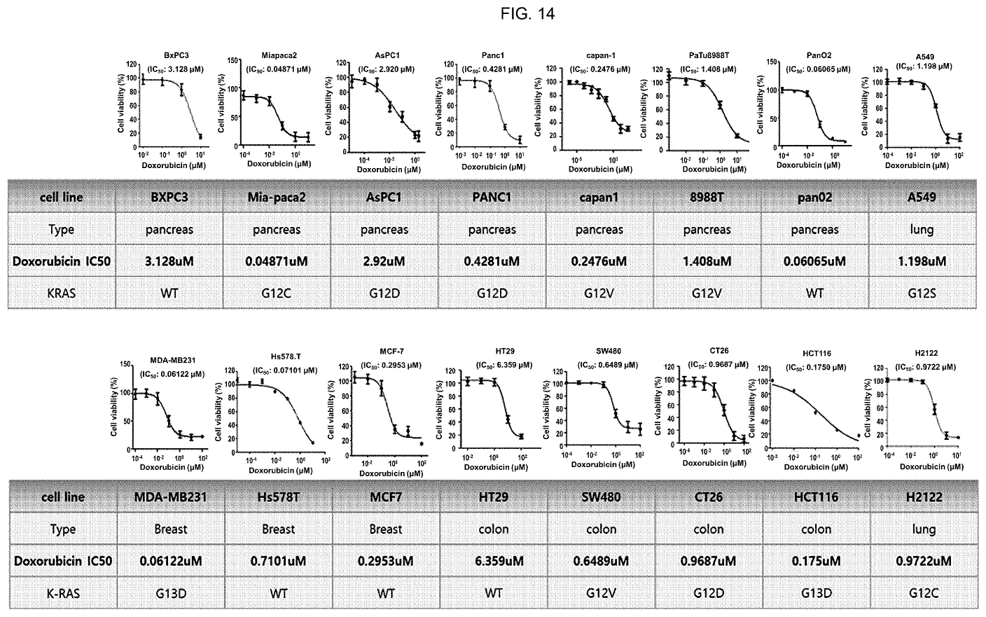

The maleimide-KGDEVD-PABC-doxorubicin conjugate according to one embodiment of the present invention (KGDEVD corresponds to SEQ ID NO: 32) was tested by MTT analysis at increasing concentrations up to 100 M in tumor cell lines with wild-type KRAS genotype (pancreatic cancer: BXPC3, Pan02; prostate cancer: DU145; colorectal cancer: HT29; breast cancer: Hs578T, MCF7) and those with KRAS variant genotype (pancreatic cancer: AsPC3, Mia-paca2, Capan-1, panC1, 8988T; prostate cancer: PC3; colorectal cancer: CT26, HCT116, SW480; breast cancer: MDA-B231; lung cancer: H2122, A549) (FIG. 14).

As a result, as shown in FIG. 14, the IC₅₀ of the prodrug conjugate according to one embodiment of the present invention was generally higher in the cancer cells with wild-type KRAS genotype, whereas it was lower in those with KRAS variant genotype.

### Experimental Example 12: Evaluation of Cytotoxicity at Different Concentrations of Malamide-KGDEVD-PABC-Doxorubicin in Tumor Cells with PTEN Protein Loss Genotype And Normal Tumor Cells

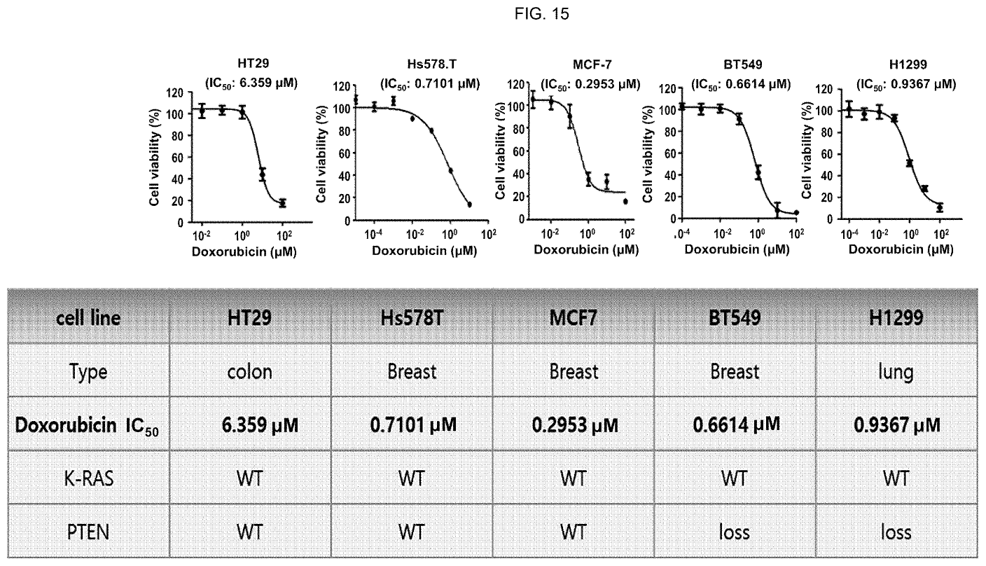

The present inventors conducted a MTT analysis on the maleimide-KGDEVD-PABC-doxorubicin conjugate synthesized in Example 1 (KGDEVD corresponds to SEQ ID NO: 32) in tumor cell lines with wild-type PTEN protein genotype (prostate cancer: DU145; colon cancer: HT29; breast cancer: Hs578T, MCF7) and those with PTEN protein loss genotype (colon cancer: HT29; breast cancer: BT579, MDA-MB436; lung cancer: H1299) at increasing concentrations up to 100 μM (FIG. 15).

As a result, as shown in FIG. 15, the cancer cells with PTEN wild-type genotype exhibited lower IC₅₀ values based on doxorubicin, while the tumor cell lines with PTEN protein loss genotype exhibited higher IC₅₀ values.

### Experimental Example 13: Tumor Growth Profile after Administration of Maleimide-KGDEVD-PABC-Doxorubicin to Mice Bearing Tumors with and without KRAS Variant Genotype

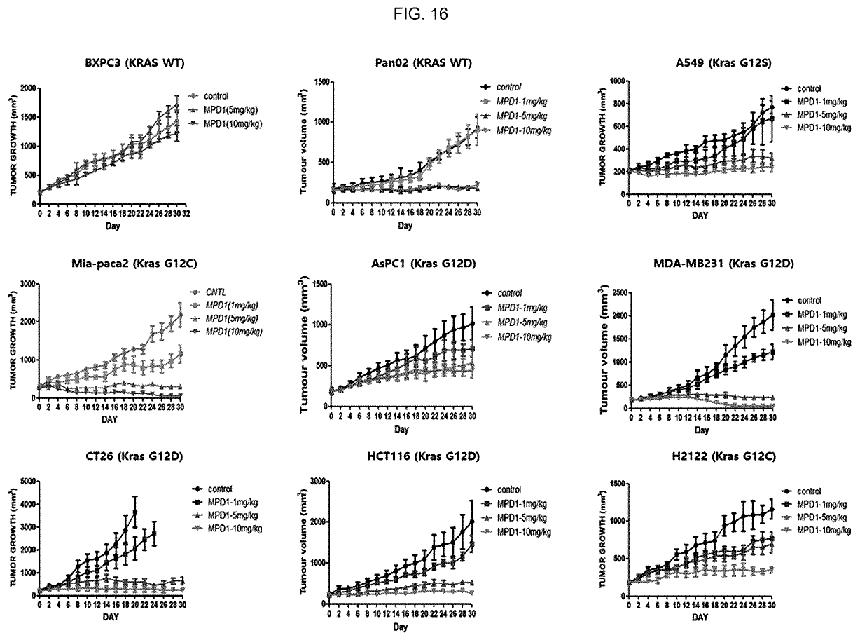

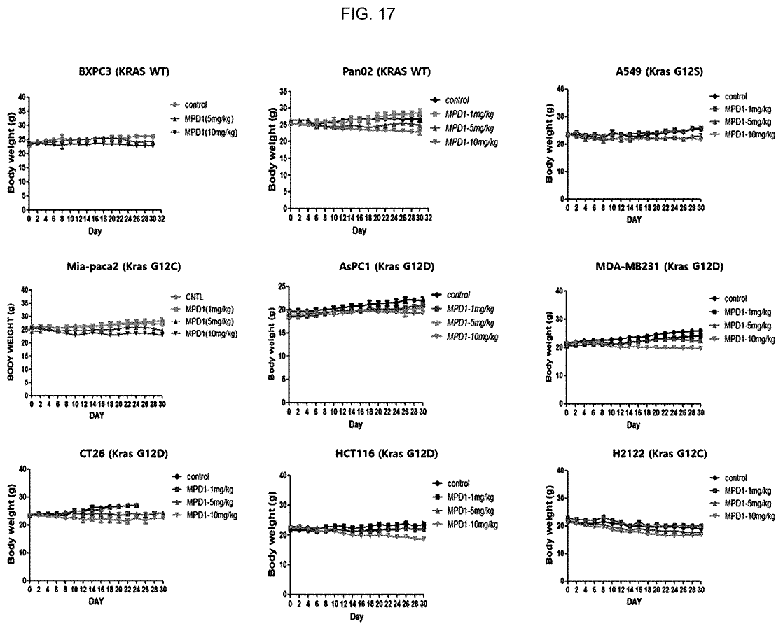

The present inventors evaluated the extent of tumor growth inhibition by the maleimide-KGDEVD-PABC-doxorubicin synthesized in Example 1 (KGDEVD corresponds to SEQ ID NO: 32) using tumor-induced C3H/HeN mice models, which were inoculated with cell lines with wild-type KRAS genotype (pancreatic cancer: BXPC3, Pan02) and those with KRAS variant genotype (pancreatic cancer: Mia-paca2, AsPC1; breast cancer: MDA-MB231; colon cancer: CT26, HCT116; lung cancer: H2122, A549) (FIG. 16 and FIG. 17). The maleimide-KGDEVD-PABC-doxorubicin was administered intravenously every 3 days at doses equivalent to 1, 5, and 10 mg/kg of doxorubicin, respectively, and then observed for 1 month. As shown in FIG. 16, the results showed that the cancer cells with KRAS variant genotype exhibited better tumor growth inhibition than those with wild-type KRAS genotype. As shown in FIG. 17, none of the drugs had a significant effect on the body weight of the experimental animals. Therefore, the prodrug conjugate according to one embodiment of the present invention is not expected to have significant side effects.

### Experimental Example 14: Examination of Tumor Growth Profile after Administration Of Maleimide-KGDEVD-PABC-Doxorubicin to Mice Bearing Tumors with and without PTEN Protein Loss Genotype

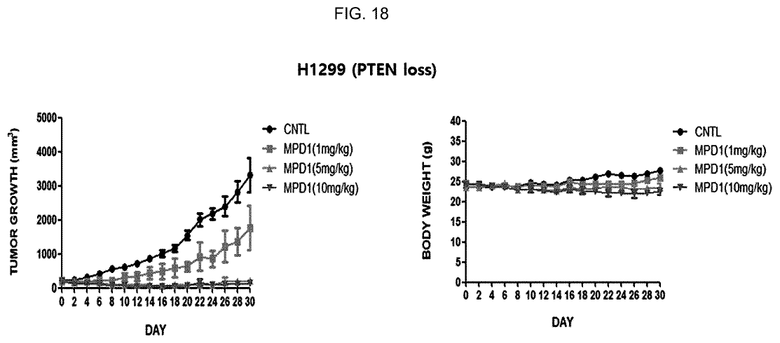

The present inventors evaluated the extent of tumor growth inhibition by the maleimide-KGDEVD-PABC-doxorubicin synthesized in Example 1 (KGDEVD corresponds to SEQ ID NO: 32) using tumor-induced C3H/HeN mice models, which were inoculated with cancer cell groups with PTEN protein loss genotype (lung cancer: H1299; breast cancer: MDA-MB436) (FIG. 18). The maleimide-KGDEVD-PABC-doxorubicin was administered intravenously every 3 days at concentrations equivalent to 1, 5, and 10 mg/kg of doxorubicin, respectively, and observed for 1 month. As shown in FIG. 18, the results demonstrated greater tumor inhibition in the experimental group inoculated with the cancer cells with PTEN protein loss genotype compared to the control group.

### Experimental Example 15: Comparison of Post-Dose Tumor Growth Profiles Between Maleimide-KGDEVD-PABC-Doxorubicin, Maleimide-KGDVED-PABC-Doxorubicin with Different Peptide Sequence, and Conventional Drug (Aldoxorubicin) in Tumor Cells with KRAS Variant Genotype

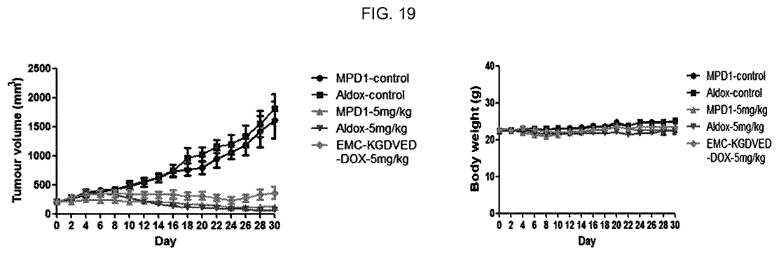

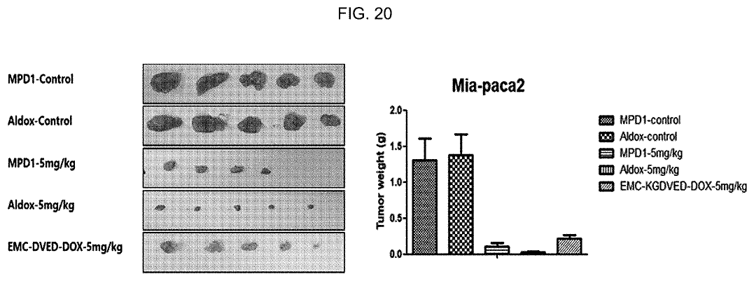

Using C3H/HeN mice implanted with tumor cells having KRAS variant genotype (Mia-paca2), the tumor inhibition effect of the maleimide-KGDEVD-PABC-doxorubicin (MPD1) synthesized in Example 1 of the present invention, the maleimide-KGDVED-PABC-doxorubicin (EMC-KGDVED-DOX) with different peptide sequence, and Aldoxorubicin, a conventional doxorubicin-based anticancer drug which is linked to a maleimide so that it binds to albumin in plasma upon administration and increases half-life, were compared (FIGS. 19 and 20). (KGDEVD corresponds to SEQ ID NO: 32) These prodrug conjugates and anticancer drug were administered intravenously at an amount equivalent to 5 mg/kg based on doxorubicin every 3 days and observed for 1 month.

As a result, as shown in FIGS. 19 and 20, the doxorubicin prodrug conjugate according to one embodiment of the present invention exhibited anticancer effects similar to those of Aldoxorubicin, the conventional maleimide-adducted doxorubicin-based anticancer drug. This was expected given the similarity between the mechanism of action of Aldoxorubicin and the prodrug conjugate of the present invention. The maleimide-KGDVED-PABC-doxorubicin which has a peptide linker not cleavable by caspase, showed very good in vivo anticancer effects, although slightly less than the maleimide-KGDEVD-PABC-doxorubicin synthesized in Example 1 and Aldoxorubicin. This suggests that once the prodrug conjugate according to one embodiment of the present invention is taken up by cancer cells with KRAS variant genotype, it is cleaved by various exoproteases through intracellular mechanisms other than that by caspase, e.g., the endosomal-lysosomal pathway, which results in the release of free doxorubicin that exerts the anticancer effect. This implies that for cancers with KRAS variant genotype or PTEN protein loss genotype, it is not necessary to use peptide linkers that are recognized by cancer cell-specific protein cleavage enzymes such as caspases, but prodrugs where albumin binding moieties are linked to anticancer chemotherapeutic agents using various peptide linkers and/or chemical linkers may be sufficiently effective against cancer.

The present invention has been described with reference to the embodiments and experimental examples described above, but these are exemplary only, and one having ordinary skill in the art will understand that various modifications and equally other embodiments or experimental examples are possible from them. The true scope of technical protection of the invention should therefore be determined by the technical ideas of the appended claims of the patent.

### INDUSTRIAL AVAILABILITY

The prodrug conjugate according to one embodiment of the present invention may be useful as an anticancer agent.

1-10. (canceled)

11. A method of selecting a cancer patient for treatment with an anticancer prodrug conjugate, comprising:
isolating DNA or protein from a cancer tissue biopsy obtained from the cancer patient;
identifying from the isolated DNA or protein one or more of (i) a genotype of the patient's gene encoding KRAS, (ii) a genotype of the patient's gene encoding PTEN protein, and/or investigating loss of expression of PTEN protein using; and,
selecting the cancer patient for treatment by administration of an anticancer prodrug conjugate comprising an albumin binding moiety, linker, and anticancer compound if one or more of (i) the patient's gene encoding KRAS has been mutated, ii the patient's gene encoding PTEN protein has been mutated such that it causes a loss of PTEN protein, and (iii) loss of PTEN protein expression is identified.

12. The method according to claim 11, wherein the method comprises analyzing the patient's gene encoding KRAS by sequencing, DNA microarray, or allele-specific PCR reaction to determine whether the patient's gene encoding KRAS has been mutated.

13. The method according to claim 11, wherein the method comprises one or both of genotyping the patient's gene encoding PTEN protein and quantitative analysis of PTEN protein expression to determine whether loss of PTEN protein expression has occurred.

14. A method of treating cancer in a cancer patient with one or both of a KRAS variant and loss of PTEN protein expression, comprising
isolating DNA or protein from a cancer tissue biopsy obtained from the cancer patient;
identifying from the isolated DNA or protein one or more of (i) a genotype of the patient's gene encoding KRAS, (ii) a genotype of the patient's gene encoding PTEN protein, and/or investigating loss of expression of PTEN protein; and,
administering a therapeutically effective amount of an anticancer prodrug conjugate comprising an albumin binding moiety, linker, and anticancer compound to the patient if one or more of (i) the patient's gene encoding KRAS has been mutated, ii) the patient's gene encoding PTEN protein has been mutated such that it causes a loss of PTEN protein, and (iii) loss of PTEN protein expression is identified.

15. The method of treatment according to claim 14, wherein the method comprises analyzing of the patient's gene encoding KRAS by sequencing, DNA microarray, or allele-specific PCR reaction to determine whether the patient has a KRAS variant.

16. The method of treatment according to claim 14, wherein the method comprises one or both of genotyping the patient's gene encoding PTEN protein and quantitative analysis of PTEN protein expression to identify loss of PTEN protein expression.

17. (canceled)

18. The method of claim 11, wherein the albumin binding moiety of the anticancer prodrug conjugate is selected from a maleimide group, a pyridyldithiol group, an oleate group, polyethylene glycol, hyaluronic acid, an albumin binding peptide (PEP, SEQ ID NO: 1), a palmitate group, 4-(p-iodophenyl)butyric acid, or a single-chain based antibody analogue that specifically binds to albumin, such as V_{H}H, scFv, V_{NAR}, DARPin, nanobody, monobody, or VLR.

19. The method of claim 11, wherein the linker of the anticancer prodrug conjugate is a peptide linker, a non-peptide linker, or a linker in the form of a combination of a peptide linker and a non-peptide linker.

20. The method of claim 19, wherein the linker is an in vivo cleavable peptide linker or an in vivo non-cleavable linker.

21. The method of claim 20, wherein the linker is an in vivo cleavable peptide linker that is a cyclopeptide peptide linker or a protease-sensitive peptide linker.

22. The method of claim 21, wherein the linker is a protease-sensitive peptide linker that comprises a peptide that is cleaved by caspase, cathepsin, purine, or matrix metalloprotease.

23. The method of claim 21, wherein the linker is a protease-sensitive peptide linker that comprises one or more than two amino acid sequences selected from the group consisting of DEVD (SEQ ID NO: 13), DLDV (SEQ ID NO: 14), DEID (SEQ ID NO: 15), DEHD (SEQ ID NO: 16), DKAD (SEQ ID NO: 17), DSFD (SEQ ID NO: 18), DSSD (SEQ ID NO: 19), DGKD (SEQ ID NO: 20), DYND (SEQ ID NO: 21), DRPD (SEQ ID NO: 22), DNVD (SEQ ID NO: 23), VQVD (SEQ ID NO: 24), LETD (SEQ ID NO: 25), LEHD (SEQ ID NO: 26), WEHD (SEQ ID NO: 27), ELQTDG (SEQ ID NO: 28), RIEADS (SEQ ID NO: 29), VDVAD (SEQ ID NO: 30), DFRD (SEQ ID NO: 31), KGDEVD (SEQ ID NO: 32), RGDEVD (SEQ ID NO: 33), CRGDCGGDEVD (SEQ ID NO: 34), DEVDR (SEQ ID NO: 35), CQRPPRDEVD (SEQ ID NO: 36), GRRG (SEQ ID NO: 37), FRRG (SEQ ID NO: 38), ARRG (SEQ ID NO: 39), KGRRG (SEQ ID NO: 40), RGDRRG (SEQ ID NO: 41), DXXD (SEQ ID NO: 42), LXXD (SEQ ID NO: 43), and VXXD (SEQ ID NO: 44).

24. The method of 19, wherein the linker is in the form of a combination of a peptide linker and non-peptide linker selected from KGDEVD-PABC, DEVD-PABC, RGDEVD-PABC, RGDEVD-MBA, CQRPPRDEVD-PABC, DEID-PABC, DLVD-PABC, RGDEVD-MBA, and KGDEVD-PABC, wherein KGDEVD corresponds to SEQ ID NO: 32, DEVD corresponds to SEQ ID NO: 13, RGDEVD corresponds to SEQ ID NO: 33, CQRPPRDEVD corresponds to SEQ ID NO: 36, DEID corresponds to SEQ ID NO: 15, and DLVD corresponds to SEQ ID NO: 14.

25. The method of claim 11, wherein the chemotherapeutic agent is selected from the group consisting of cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, and floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, and derivatives thereof.

26. The method of claim 11, wherein the anticancer prodrug conjugate is selected from the group consisting of maleimide-KGDEVD-PABC-doxorubicin, maleimide-KGDEVD-PABC-daunorubicin, maleimide-KGDEVD-PABC-paclitaxel, maleimide-KGDEVD-PABC-MMAE, Maleimide-DEVD-PABC-doxorubicin, Maleimide-DEID-PABC-doxorubicin, Maleimide-DLVD-PABC-doxorubicin, Maleimide-DEVD-doxorubicin, Pyridyldithiol-KGDEVD-PABC-doxorubicin, Oleate-KGDEVD-PABC-Doxorubicin, Polyethylene Glycol-KGDEVD-PABC-Doxorubicin, Hyaluronan-KGDEVD-PABC-Doxorubicin, Folate-KGDEVD-PABC-Doxorubicin, RGDEVD-PABC-Doxorubicin, CQRPPRDEVD-PABC-doxorubicin, RGDEVD-MBA-doxorubicin, DEVD-daunorubicin-RGDSC, and HSA-maleimide-KGDEVD-PABC-doxorubicin, wherein KGDEVD corresponds to SEQ ID NO: 32, DEVD corresponds to SEQ ID NO: 13, DEID corresponds to SEQ ID NO: 15, DLVD corresponds to SEQ ID NO: 14, RGDEVD corresponds to SEQ ID NO: 33, and CQRPPRDEVD corresponds to SEQ ID NO: 36.

27. The method of claim 14, wherein the albumin binding moiety of the anticancer prodrug conjugate is selected from a maleimide group, a pyridyldithiol group, an oleate group, polyethylene glycol, hyaluronic acid, an albumin binding peptide (PEP, SEQ ID NO: 1), a palmitate group, 4-(p-iodophenyl)butyric acid, or a single-chain based antibody analogue that specifically binds to albumin, such as V_{H}H, scFv, V_{NAR}, DARPin, nanobody, monobody, or VLR.

28. The method of claim 14, wherein the linker of the anticancer prodrug conjugate is a peptide linker, a non-peptide linker, or a linker in the form of a combination of a peptide linker and non-peptide linker.

29. The method of claim 28, wherein the linker is an in vivo cleavable peptide linker or an in vivo non-cleavable linker.

30. The method of claim 29, wherein the linker is an in vivo cleavable peptide linker that is a cyclopeptide peptide linker or a protease-sensitive peptide linker.

31. The method of claim 30, wherein the linker is a protease-sensitive peptide linker that comprises a peptide that is cleaved by caspase, cathepsin, purine, or matrix metalloprotease.

32. The method of claim 30, wherein the linker is a protease-sensitive peptide linker that comprises one or more than two amino acid sequences selected from the group consisting of DEVD (SEQ ID NO: 13), DLDV (SEQ ID NO: 14), DEID (SEQ ID NO: 15), DEHD (SEQ ID NO: 16), DKAD (SEQ ID NO: 17), DSFD (SEQ ID NO: 18), DSSD (SEQ ID NO: 19), DGKD (SEQ ID NO: 20), DYND (SEQ ID NO: 21), DRPD (SEQ ID NO: 22), DNVD (SEQ ID NO: 23), VQVD (SEQ ID NO: 24), LETD (SEQ ID NO: 25), LEHD (SEQ ID NO: 26), WEHD (SEQ ID NO: 27), ELQTDG (SEQ ID NO: 28), RIEADS (SEQ ID NO: 29), VDVAD (SEQ ID NO: 30), DFRD (SEQ ID NO: 31), KGDEVD (SEQ ID NO: 32), RGDEVD (SEQ ID NO: 33), CRGDCGGDEVD (SEQ ID NO: 34), DEVDR (SEQ ID NO: 35), CQRPPRDEVD (SEQ ID NO: 36), GRRG (SEQ ID NO: 37), FRRG (SEQ ID NO: 38), ARRG (SEQ ID NO: 39), KGRRG (SEQ ID NO: 40), RGDRRG (SEQ ID NO: 41), DXXD (SEQ ID NO: 42), LXXD (SEQ ID NO: 43), and VXXD (SEQ ID NO: 44).

33. The method of 28, wherein the linker is in the form of a combination of a peptide linker and a non-peptide linker selected from KGDEVD-PABC, DEVD-PABC, RGDEVD-PABC, RGDEVD-MBA, CQRPPRDEVD-PABC, DEID-PABC, DLVD-PABC, RGDEVD-MBA, and KGDEVD-PABC, wherein KGDEVD corresponds to SEQ ID NO: 32, DEVD corresponds to SEQ ID NO: 13, RGDEVD corresponds to SEQ ID NO: 33, CQRPPRDEVD corresponds to SEQ ID NO: 36, DEID corresponds to SEQ ID NO: 15, and DLVD corresponds to SEQ ID NO: 14.

34. The method of claim 14, wherein the chemotherapeutic agent is selected from the group consisting of cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, and floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, and derivatives thereof.

35. The method of claim 14, wherein the anticancer prodrug conjugate is selected from the group consisting of maleimide-KGDEVD-PABC-doxorubicin, maleimide-KGDEVD-PABC-daunorubicin, maleimide-KGDEVD-PABC-paclitaxel, maleimide-KGDEVD-PABC-MMAE, Maleimide-DEVD-PABC-doxorubicin, Maleimide-DEID-PABC-doxorubicin, Maleimide-DLVD-PABC-doxorubicin, Maleimide-DEVD-doxorubicin, Pyridyldithiol-KGDEVD-PABC-doxorubicin, Oleate-KGDEVD-PABC-Doxorubicin, Polyethylene Glycol-KGDEVD-PABC-Doxorubicin, Hyaluronan-KGDEVD-PABC-Doxorubicin, Folate-KGDEVD-PABC-Doxorubicin, RGDEVD-PABC-Doxorubicin, CQRPPRDEVD-PABC-doxorubicin, RGDEVD-MBA-doxorubicin, DEVD-daunorubicin-RGDSC, and HSA-maleimide-KGDEVD-PABC-doxorubicin, wherein KGDEVD corresponds to SEQ ID NO: 32, DEVD corresponds to SEQ ID NO: 13, DEID corresponds to SEQ ID NO: 15, DLVD corresponds to SEQ ID NO: 14, RGDEVD corresponds to SEQ ID NO: 33, and CQRPPRDEVD corresponds to SEQ ID NO: 36.
